(19) United States
(12) Patent Application Publication
(10) Pub. No.: US 2018/0340943 A1
(43) Pub. Date: Nov. 29, 2018

(54) Identification of Biomarkers in Serum Samples Collected from Clinical Trials by Multiplex Technology

(71) Applicant: Sirbal Ltd., Limassol (CY)

(72) Inventors: Nadav SHRAIBOM, Herzelia (IL); Anu T SINGH, Noida (IN); Manu JAGGI, DLF City (IN); Eran STEINGERG, San Francisco, CA (US); Ritu VERMA, Ghaziabad (IN); Alka MADAAN, Ghaziabad (IN)

(21) Appl. No.: 15/778,244

(22) PCT Filed: Nov. 23, 2016
(86) PCT No.: PCT/US16/63706

§ 371 Date: May 23, 2018

Related U.S. Application Data
(63) Continuation-in-part of application No. 15/197,740, filed on Jun. 29, 2016.
Application No. 15/197,740 is a continuation-in-part of application No. 15/131,743, filed on Apr. 18, 2016.
Application No. 15/131,743 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
Application No. PCT/US16/63706 is a continuation-in-part of application No. 15/197,745, filed on Jun. 29, 2016.
Application No. 15/197,745 is a continuation-in-part of application No. 15/131,743, filed on Apr. 18, 2016.
Application No. 15/131,743 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
(60) Provisional application No. 62/259,056, filed on Nov. 23, 2015.
(60) Provisional application No. 62/268,226, filed on Dec. 16, 2015.
(60) Provisional application No. 62/355,614, filed on Jun. 28, 2016.
(60) Provisional application No. 62/198,637, filed on Jul. 29, 2015.
(60) Provisional application No. 62/297,796, filed on Feb. 19, 2016.
(60) Provisional application No. 62/313,709, filed on Mar. 26, 2016.
(60) Provisional application No. 62/325,993, filed on Apr. 21, 2016.
(60) Provisional application No. 62/348,762, filed on Jun. 10, 2016.
(60) Provisional application No. 62/355,614, filed on Jun. 28, 2016.
(60) Provisional application No. 62/259,056, filed on Nov. 23, 2015.
(60) Provisional application No. 62/268,226, filed on Dec. 16, 2015.
(60) Provisional application No. 62/198,637, filed on Jul. 29, 2015.
(60) Provisional application No. 62/297,796, filed on Feb. 19, 2016.
(60) Provisional application No. 62/313,709, filed on Mar. 26, 2016.
(60) Provisional application No. 62/325,993, filed on Apr. 21, 2016.
(60) Provisional application No. 62/348,762, filed on Jun. 10, 2016.
(60) Provisional application No. 62/355,614, filed on Jun. 28, 2016.
(60) Provisional application No. 62/259,056, filed on Nov. 23, 2015.
(60) Provisional application No. 62/268,226, filed on Dec. 16, 2015.

(51) Int. Cl. G01N 33/68; A61K 36/708; A61K 36/64; A61K 36/355; A61K 9/00

(52) U.S. Cl. CPC G01N 33/6869; G01N 33/6863; A61K 36/708; A61K 36/64; G01N 2800/205; A61K 9/0014; G01N 2333/5437; G01N 2333/54; G01N 2333/535; A61K 36/355

(57) ABSTRACT

The present invention provides methods, compositions, and kits for the treatment and/or diagnosis of psoriasis. The invention includes a method for diagnosing psoriasis, comprising measuring a level or combination of levels of one or more of: IL-5, IL-6, IL-7, IL-10, IL-13, IL-33, FGF-B, GMCSF, VEGF, IL-33, MCP-1, or IP-10, or combinations thereof, in a sample from a patient; comparing said measured level or combination of levels; and correlating said level or combination of levels with a manifestation of psoriasis within said patient. A medicinal composition comprising an herbal combination of Da Huang, Sheng Di Huang, and Jin Yin Hua for the treatment of psoriasis in a patient. A kit for diagnosing and/or prognosing psoriasis in a patient.

### PRIORITY AND RELATED APPLICATIONS

This application claims priority to U.S. provisional patent application Ser. No. 62/259,056, filed Nov. 23, 2015; U.S. Ser. No. 62/268,226, filed Dec. 16, 2015; and U.S. Ser. No. 62/355,614, filed Jun. 28, 2016, which are each incorporated by reference.

This application is also a Continuation in Part (CIP) of Ser. No. 15/197,740, filed Jun. 29, 2016; which is a continuation-in-part (CIP) of U.S. patent application Ser. No. 15/131,743, filed Apr. 18, 2016; which claims priority to U.S. Ser. No. 62/198,637, filed Jul. 29, 2015; and U.S. Ser. No. 62/297,796, filed Feb. 19, 2016; and which is a CIP of U.S. Ser. No. 14/754,266 filed Jun. 29, 2015; which is a CIP of U.S. Ser. No. 13/900,525, filed May 22, 2013, now U.S. Pat. No. 9,095,606; and which claims priority to U.S. Ser. No. 62/313,709, filed Mar. 26, 2016; and to U.S. Ser. No. 62/325,993, filed Apr. 21, 2016; and to U.S. Ser. No. 62/348,762, filed Jun. 10, 2016; and which claims priority to U.S. Ser. No. 62/355,614, filed Jun. 28, 2016; U.S. Ser. No. 62/259,056, filed Nov. 23, 2015; and to U.S. Ser. No. 62/268,226, filed Dec. 16, 2015.

This application is also a Continuation in Part (CIP) of Ser. No. 15/197,745, filed Jun. 29, 2016; which is a continuation-in-part (CIP) of U.S. patent application Ser. No. 15/131,743, filed Apr. 18, 2016; which claims priority to U.S. Ser. No. 62/198,637, filed Jul. 29, 2015; and U.S. Ser. No. 62/297,796, filed Feb. 19, 2016; and which is a CIP of U.S. Ser. No. 14/754,266 filed Jun. 29, 2015; which is a CIP of U.S. Ser. No. 13/900,525, filed May 22, 2013, now U.S. Pat. No. 9,095,606; and which claims priority to U.S. Ser. No. 62/313,709, filed Mar. 26, 2016; and to U.S. Ser. No. 62/325,993, filed Apr. 21, 2016; and to U.S. Ser. No. 62/348,762, filed Jun. 10, 2016; and which claims priority to U.S. Ser. No. 62/355,614, filed Jun. 28, 2016; U.S. Ser. No. 62/259,056, filed Nov. 23, 2015; and to U.S. Ser. No. 62/268,226, filed Dec. 16, 2015.

This application is related to U.S. patent application Ser. No. 15/131,743, filed Apr. 18, 2016, U.S. Ser. No. 62/198,637, filed Jul. 29, 2015, U.S. Ser. No. 62/297,796, filed Feb. 19, 2016, U.S. Ser. No. 62/313,709, filed Mar. 26, 2016, U.S. Ser. No. 62/325,993, filed Apr. 21, 2016, U.S. Ser. No. 62/348,762, filed Jun. 10, 2016, and to U.S. patent application Ser. Nos. 15/133,056, 14/754,266, PCT/US15/38341, 14/710,865, 14/815,892, 14/287,158, 14/287,153, 13/890,990, PCT/US13/72670, 14/981,899, 14/815,705, 13/152,039, PCT/US11/60501, and 61/413,430; and to US published patent applications nos. 20160113982 A1, 20160051553 A1, 20160136220 A1, 20160136219 A1, 20160136216 A1, 20160136223 A1, 20160136222 A1, 20160136221 A1, 20160113983 A1, 20160143980 A1, 20160136218 A1, 20140205685 A1, and 20140206631 A1; and to U.S. Pat. Nos. 9,066,974, 9,095,606, 8,734,859, 8,597,695 and 8,541,382, all of which are incorporated by reference.

### BACKGROUND OF THE INVENTION

### 1. Field of the Invention

The present invention relates generally to diagnostic testing and test kits for psoriasis and to prognostic testing and test kits for psoriasis treatments including herbal and/or molecular combinations.

### 2. Description of the Related Art

Psoriasis is a chronic inflammatory, multi-system disease associated with considerable morbidity and co-morbid conditions. Psoriasis affects approximately 2% of the world's population. The severity of psoriasis is defined by extent of body surface area involvement, involvement of localized body regions such as flexures, hands, feet, facial, and genital regions which, may interfere significantly with activities of daily life and have a substantial psychological impact on one's personal well-being. In psoriasis, the immune system is mistakenly activated, which leads to overproduction of skin cells. Skin cells build up too rapidly on the surface of the skin, forming raised, red, scaly patches (called plaques). These plaques are often itchy and sometimes painful. The causes of psoriasis are not fully understood today. The current consensus is that immune system, genetics and the environment (e.g., stress, cold weather) all play major roles in the development of psoriasis. As a result there is skin inflammation accompanied by overproduction of skin cells. Cells in the upper skin layer normally mature and are shed from the skin's surface every 28 to 30 days. With psoriasis, the cells can mature in 3 to 6 days then move to the skin and pile up.

Psoriasis (Greek, psora, “itch” and -iasis, “action, condition”) is a chronic inflammatory and autoimmune skin disorder, which is characterized by hyper-proliferation and abnormal differentiation of keratinocytes, increased infiltration of leukocytes and aggravated inflammatory conditions (Schon, 2005). The onset and development of psoriasis involves a complex interplay of multiple genetic and environmental factors. This skin disorder is established as a multifactorial disease, which is reported to affect 2-3% of the worldwide population (Parisi et al., 2013). It is primarily characterized by presence of red, itchy, flaky and crusty patches of skin, covered with silvery scales, most commonly located over the scalp surfaces, around or in the ears, elbows, knees, navel, genitals and buttocks or involvement of almost the entire body surface is also observed (Lebwohl, 2003; Nickoloff and Nestle, 2004). Formation of inflamed and raised plaques on the skin, that constantly shed the scales derived from excessive growth of skin-epithelial cells is a distinct feature of psoriasis.

Although the causative antigenic triggers involved in the etio-pathogenesis of psoriasis are not yet completely understood, the crosstalk between keratinocyte arm and the immune arm has been considered pivotal in investigating the mechanism of action and efficacy of potential anti-psoriatic drugs (Lowes et al., 2007). Pharmacological agents that simultaneously target the hyper-proliferation along-with apoptosis arm or inflammation arm, or a combination of these two together have emerged as efficacious drugs for management of psoriasis (Chakrabarti et al., 2012).

The hyperproliferative tendency with abnormal differentiation of keratinocytes in epidermis is established as an integral cellular event underlying the pathogenesis of psoriasis. Anti-psoriatic compounds specifically targeting the hyper-proliferation of keratinocytes are of special interest, since this action may lead to restoration of the perturbed balance and homeostatic control of abnormal growth of keratinocytes and recovery from psoriatic conditions to normal skin (Pol et al., 2003). This class of drugs includes anthracenones derivatives (Muller et al., 2001), artemether (Wu et al., 2015), benzodiazepine derivatives (Bhagavathula et al., 2008), fumaric acid derivatives (Sebok et al., 1994), indigo naturalis (Lin et al., 2009), lapacho compounds (Muller et al., 1999) and Rubia cordifolia L extract (Lin et al., 2010).

Apoptosis helps in the efficient removal of dysfunctional cells without generation of an inflammatory response and hence it is considered as a crucial event in maintenance of homeostasis in continually renewing tissues such as skin (Bianchi L et al., 1994; Tse et al., 2008). The regulatory switch balancing the turnover of keratinocytes determined by proliferation and controlled apoptosis is disturbed in psoriatic conditions. In psoriatic lesions, resistance of epidermal keratinocytes to undergo apoptosis is known to primarily support their hyper-proliferation (Wrone-Smith et al., 1997). Any disturbance in apoptotic mechanisms results in hyper-proliferation of keratinocytes and onset of psoriasis (Kawashima et al., 2004). Most of the drugs inhibiting keratinocyte proliferation growth exert their anti-proliferative action via induction of apoptotic cascades, such as anthralin (McGill et al., 2005), arsenic derivatives (Tse et al., 2008), isocamptothecin (Lin et al., 2008), topical corticosteroids (Guichard et al., 2015), 1,4-dihydroxy-2-naphthoic Acid (Mok et al., 2013), Realgar (Tse et al., 2009) and Tanshinone IIA (Li et al., 2012).

Inflammation is another crucial arm in the pathogenesis of psoriasis (Cai Y et al., 2012). A complex interaction of activated type-1 T-lymphocytes along with antigen-presenting cells and keratinocytes (cell populations that secrete excessive pro-inflammatory molecules) is the key-determining step for hyperproliferation and a marked inflammation in epidermis as well as dermis (Zampetti A et al., 2009). Enhanced secretion of pro-inflammatory cytokines such as TNF-α, IFN-γ, and IL-6 and other key mediators such as NO, sPLA₂ secreted by keratinocytes contribute towards aggravation of inflammation associated with psoriasis. This in-turn promotes an enhanced infiltration of immune cell populations, such as macrophages, neutrophils and dendritic cells towards the inflammatory site, resulting in further secretion of cytokines, activation of keratinocytes to undergo aberrant hyper-proliferation and differentiation and aids in development of psoriasis (Marble et al., 2007). Anti-inflammatory activity of anti-psoriatic drugs in HaCaT cells have been reported for Leutolin (Weng et al., 2014), Jakyakgamcho-tang (Paeonialactiflora and Glycyrrhizauralensis) (Jeong et al., 2015), Banhasasim-tang (BHSST) (Jin et al., 2015) and in immune cells for Avarol (Amigo et al., 2007, 2008), Apremilast (Schafer et al., 2010) and Isothiocyanates (Yehuda et al., 2012).

Angiogenesis is another key arm, which plays a supporting role in development of psoriasis. With early psoriatic events, increased formation of new dermal blood vessels is initiated and subsequently disappears with disease clearance (Heidenreich et al., 2009). The expansion of dermal microvascular system in the psoriatic lesions suggests that psoriasis is an angiogenesis-dependent disorder (Barker, 1991). Vascular endothelial growth factor (VEGF) is a well-established mediator of angiogenesis, which is over-secreted by epidermal keratinocytes located in the inflammatory psoriatic lesions and endothelial cells (Detmar et al., 1994). Hence, targeting the angiogenesis arm and VEGF secretion has emerged out as a novel therapeutic strategy for psoriasis (Li et al., 2014).

Herbal medicines are prevalent, and serve the medicinal needs of a large population around the world. The global herbal medicine market is currently worth around $30 billion. There is also an increased effort for the isolation of bioactive phytochemicals from herbs for their possible usefulness in the control of various ailments.

It is desired to provide more reliable medical kits that include diagnostic test kits and medicines for treating a psoriasis patient in accordance with an indication provided by the test kit.

It is desired to provide more reliable prognostic kits that include psoriasis medicines and prognostic measuring devices and prognostic indicators. With such prognostic kits, more effective stratification of psoriasis patients is provided for certain treatments for certain conditions such that patients can be treated more safely and effectively.

It is therefore desired to provide diagnostic test kits configured to measure levels of certain panels of one or more diagnostic markers that are most indicative of a psoriasis patient's disposition with a disease, condition or disorder, or combination or ailments.

It is also desired to provide prognostic test kits configured to measure levels of certain panels of one or more prognostic markers that are most indicative of a psoriasis patient's tendency to respond safely and effectively to certain treatments or combinations of treatments for certain diseases, conditions or disorders.

It is also desired to have an herbal and/or molecular combination that may be administered to a psoriasis patient as a safe and effective treatment of a condition.

It is also desired to have an herbal and/or molecular combination that may be administered to a psoriasis patient before, during and/or after a typical, known or discovered treatment regimen to enhance the effectiveness of such known or discovered treatments, and/or to reduce side effects of such known or discovered treatments and/or for weening a patient from a dependence on such known or discovered treatments.

### BRIEF DESCRIPTION OF THE DRAWINGS

FIG. 1A illustrates an antipsoriatic mechanism for a medicinal composition including an herbal combination.

FIG. 1B illustrates a representative HPLC profile of SIRB-001, detection UV was at 230 nm and 330 nm. Chromatographic conditions are described in section 2.4 of materials and methods.

FIG. 1C illustrates a representative HPLC profile of standard marker compounds.

FIG. 2 illustrates anti-proliferative effect of SIRB-001 in keratinocytes (HaCaT cells) after 48 h of treatment. 10,000 cells/well were treated with SIRB-001. Proliferation was determined by MTT assay and expressed as a percentage (Mean±S.E.M.) of control cells. n=3. *: p<0.05, **: p<0.01, significantly different from the control group.

FIG. 3 illustrates alterations in the morphology of keratinocytes (HaCaT cells) induced by SIRB-001 after 48 h of treatment.

FIG. 4A illustrates SIRB-001 induced externalization of PS on HaCaT cell membrane by Annexin-V/7-AAD staining

FIG. 4B illustrates depolarization of mitochondrial membrane potential in HaCaT cells by SIRB-001 detected by JC-1/7-AAD staining after 24 h of treatment. 0.5×10⁶ cells/well were treated with SIRB-001 and apoptosis was analyzed using JC-1/7-AAD dual staining. Representative results demonstrate percentage population of viable/polarized cells (JC-1 Orange(+)/Green(−)), apoptotic/depolarized cells-1, (JC-1 Orange (+)/Green(+)), apoptotic/depolarized cells-2 (JC-1 Orange(−)/Green (+)) (n=3). Total 10,000 events were acquired. **: p<0.01, significantly different from the control group.

FIG. 4C illustrates Caspase-3 activation by SIRB-001 in HaCaT cells.

FIG. 4D illustrates apoptotic effect of SIRB-001 via modulation of cell cycle distribution in HaCaT cells by PI staining.

FIG. 4E illustrates SIRB-001 induced enhancement of DNA fragmentation in HaCaT cells by Hoechst.

FIGS. 5A-5F illustrate SIRB-001 induced inhibition of inflammatory markers in HaCaT cells

FIG. 6 illustrates downregulation of VEGF secretion by SIRB-001 in HaCaT cells.

FIGS. 7A-7D illustrate inhibition of IL-17/IL-23 axis by SIRB-001 in immune cells.

FIGS. 8A-8B illustrate inhibitory effects of SIRB-001 on signaling markers.

FIGS. 9A-9I illustrate effects of SIRB-001 on TPA induced ear inflammation in C57BL/6 mice.

FIGS. 10A-10D illustrate effects of SIRB-001 on IMQ induced psoriasis in Balb/c mice.

FIGS. 11A-11F schematically illustrate mechanistic action of SIRB-001 in onset, progress and development of psoriasis.

FIGS. 12A-12E schematically represent an immunoassay sandwich-based assay workflow.

FIG. 13 illustrates effects of psoriasis on levels of certain markers compared to healthy controls.

FIG. 14 illustrates effects of psoriasis on levels of certain markers compared to healthy controls.

FIG. 15 illustrate the pathogenesis of psoriasis.

FIG. 16 illustrates Th1, Th17 and Th22 cytokines in the pathogenesis of psoriasis.

FIG. 17 illustrates a microarray of several markers utilized in a diagnostic or prognostic test process in accordance with certain embodiments.

FIG. 18 illustrates % increases in biomarkers levels of psoriasis patients versus healthy controls

FIG. 19 illustrates % fold increases in biomarker levels of psoriasis patients versus healthy controls

FIG. 20 illustrates % decreases in biomarker levels after eight weeks of treatment with an herbal combination of Da Huang, Sheng Di Huang and Jin Yin Hua.

FIG. 21 illustrates the extent of normalization if certain biomarkers after eight weeks of treatment with an herbal combination of Da Huang, Sheng Di Huang and Jin Yin Hua.

### BRIEF DESCRIPTION OF THE TABLES

Table 1 is an example combination of 15 markers that may be used in a diagnostic test kit or in a prognostic test kit or both.

Table 2 is an example combination of 27 markers that may be used in a 27-plex or smaller subset within a diagnostic or prognostic kit.

Table 3 is an example combination of 35 markers that may be used in 35 plex. These 35 markers or subsets of these markers may be used in a diagnostic or prognostic test kit.

Table 4 is an example combination of 7 markers that may be measured in a diagnostic or prognostic test kit. Subsets of these 7 markers and other markers may also be used.

Table 5 is an example combination of 6 markers that may be used in a diagnostic or prognostic test kit. Subsets of these 6 markers and other markers may also be used.

### DETAILED DESCRIPTIONS OF THE EMBODIMENTS

Methods of treating psoriasis are provided. One of these methods includes measuring a level or combination of levels of one or more of: IL-5, IL-6, IL-7, IL-10, IL-13, IL-33, FGF-B, GMCSF, VEGF, IL-33, MCP-1, or IP-10, or combinations thereof, in a first bodily fluid, serum or skin sample, or both, extracted from a patient. A psoriasis diagnosis is formulated for the patient based on said measured level or combination of levels and on one or more expected correlations between said level or combination of levels and manifestation of psoriasis within said patient. A medicinal composition is administered to the patient to treat the patient in accordance with the diagnosis.

The method may include measuring a level or combination of levels of one or more of IL-5, IL-6, IL-7, IL-10, IL-13, IL-33, FGF-B, GMCSF, VEGF, IL-33, MCP-1, or IP-10, or combinations thereof, in a second bodily fluid, serum or skin sample, or both, extracted from the patient after administering the medicinal composition over a prognostic period, and indicating to repeat the administering of the medicinal composition to the patient a significant number of further times based on a comparison between the measured levels or combinations of levels within the first and second bodily fluid, serum or skin samples, or both, and on an expected correlation between certain differences between measured levels or combinations of levels in bodily fluid samples respectively extracted before and after a prognostic period of administration of the medicinal composition.

Another method of treating psoriasis includes measuring a combination of levels of IL-17, IL-23 and TNF-α in a first bodily fluid, serum or skin sample, or both, extracted from a patient and formulating a psoriasis diagnosis for the patient based on said measured level or combination of levels and on one or more expected correlations between said level or combination of levels and manifestation of psoriasis within said patient; and administering a medicinal composition to the patient to treat the patient in accordance with said diagnosis.

The method may include measuring a combination of levels of IL-17, IL-23 and TNF-α, in a second bodily fluid, serum or skin sample, or both, extracted from the patient after administering said medicinal composition over a prognostic period; and indicating to repeat the administering of the medicinal composition to said patient a significant number of further times based on a comparison between the measured levels or combinations of levels within the first and second bodily fluid, serum or skin samples, or both, and on an expected correlation between certain differences between measured levels or combinations of levels in bodily fluid samples respectively extracted before and after a prognostic period of administration of the medicinal composition.

The medicinal composition may include 20-160 mg/kg, 1.0 wt. %-15 wt. % and/or 1-12 grams of an herbal combination of Da Huang, Sheng Di Huang and Jin Yin Hua. The medicinal composition may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

The medicinal composition may be formulated as a shampoo, conditioner, cream, lotion, ointment or other topical scalp or hair treatment or as a cream, lotion, ointment or other topical skin treatment.

A psoriasis medicine is also provided. A shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, includes a predetermined number of one or more effective doses of an herbal and/or molecular combination known to decrease by 25% or more over a course of treatment a level or combination of levels of one or more of: IL-5, IL-6, IL-7, IL-10, IL-13, IL-33, FGF-B, GMCSF, VEGF, IL-33, MCP-1, or IP-10, or combinations thereof.

Another psoriasis medicine is provided. A shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses of an herbal and/or molecular combination known to decrease by 25% or more over a course of treatment a combination of levels of IL-17, IL-23 and TNF-α.

The medicine may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

The medicine may include 1.0 wt. %-15.0 wt. % of said herbal combination of Da Huang, Sheng Di Huang and Jin Yin Hua.

A medical kit is provided that includes a diagnostic kit and a medicinal formulation for diagnosing and if necessary treating psoriasis. The diagnostic kit includes a test kit and an indicator. The test kit is configured for measuring a level or combination of levels of one or more of: IL-5, IL-6, IL-7, IL-10, IL-13, IL-33, FGF-B, GMCSF, VEGF, IL-33, MCP-1, or IP-10, or combinations thereof, in a bodily fluid, serum or skin sample, or both, and the indicator is configured for providing a diagnostic result based on the measured level or combinations of levels and on one or more expected correlations between the level or combination of levels and manifestation of psoriasis. The medicinal formulation includes a shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, and includes a predetermined number of one or more effective doses of an herbal and/or molecular combination known to decrease by 25% or more over a course of treatment a level or combination of levels of one or more of: IL-5, IL-6, IL-7, IL-10, IL-13, IL-33, FGF-B, GMCSF, VEGF, IL-33, MCP-1, or IP-10, or combinations thereof.

The medicinal formulation may include 20-160 mg/kg of an herbal combination of Da Huang, Sheng Di Huang and Jin Yin Hua.

The medical kit may also include a prognostic kit including a prognostic test kit and a prognostic indicator. The prognostic test kit may be configured for measuring a level or combination of levels of one or more of: IL-5, IL-6, IL-7, IL-10, IL-13, IL-33, FGF-B, GMCSF, VEGF, IL-33, MCP-1, or IP-10, or combinations thereof, in a second bodily fluid, serum or skin sample, or both, after a prognostic period of treatment. The prognostic indicator may be configured to provide a prognostic result for the patient based on a comparison of the measured level or combination of levels in the first and second bodily fluid, serum or skin sample, or both.

Another medical kit is provided that includes a diagnostic kit and a medicinal formulation. The diagnostic kit includes a test kit and an indicator. The test kit is configured for measuring a combination of levels of two or more of IL-17, IL-23 and TNF-α, in a bodily fluid, serum or skin sample, or both. The indicator is configured to provide a diagnostic result based on the measured level or combinations of levels and on one or more expected correlations between the level or combination of levels and manifestation of psoriasis. The medicinal formulation includes a shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, and includes a predetermined number of one or more effective doses of an herbal and/or molecular combination known to decrease by 25% or more over a course of treatment a combination of levels of two or more of IL-17, IL-23 and TNF-α.

The medical kit may include a prognostic kit including a prognostic test kit and a prognostic indicator. The prognostic test kit may be configured to measure a level or combination of levels of two or more of IL-17, IL-23 and TNF-α, in a second bodily fluid, serum or skin sample, or both, after a prognostic period of treatment. The prognostic indicator may be configured to provide a prognostic result for the patient based on a comparison of the measured level or combination of levels in the first and second bodily fluid, serum or skin sample, or both.

Effective doses of the medicinal formulation may be provided that include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient, and/or 20-160 mg/kg of an herbal combination of Da Huang, Sheng Di Huang and Jin Yin Hua.

A prognostic kit is provided that includes a medicinal formulation, a prognostic test kit and an indicator. The medicinal formulation includes a shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, and includes a predetermined number of one or more effective doses of an herbal and/or molecular combination known to decrease by 25% or more over a course of treatment a level or combination of levels of one or more of: IL-5, IL-6, IL-7, IL-10, IL-13, IL-33, FGF-B, GMCSF, VEGF, IL-33, MCP-1, or IP-10, or combinations thereof. The prognostic test kit is configured to measure a level or combination of levels of one or more of IL-5, IL-6, IL-7, IL-10, IL-13, IL-33, FGF-B, GMCSF, VEGF, IL-33, MCP-1, or IP-10, or combinations thereof, in first and second bodily fluid, serum or skin samples, or both, respectively extracted from a patient before and after a prognostic period of treatment with the medicinal formulation. The prognostic indicator is configured to provide a prognostic result for the patient based on a comparison of the measured level or combination of levels in the first and second bodily fluid, serum or skin samples, or both.

Another prognostic kit is provided including a medicinal formulation, prognostic test kit and prognostic indicator. The medicinal formulation includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, and includes a predetermined number of one or more effective doses of an herbal and/or molecular combination known to decrease by 25% or more over a course of treatment a combination of levels of two or more of: IL-17, IL-23 or TNF-α, or combinations thereof. The prognostic test kit in this example is configured to measure a level or combination of levels of two or more of IL-17, IL-23 or TNF-α, or combinations thereof, in first and second bodily fluid, serum or skin samples, or both, respectively extracted from a patient before and after a prognostic period of treatment with the medicinal formulation. The prognostic indicator is configured to provide a prognostic result for the patient based on a comparison of measured levels or combinations of levels from the first and second bodily fluid, serum or skin samples, or both.

The medicinal formulation may include one or more effective doses that each include 1.0 wt. %-15.0 wt. % of an herbal combination of Da Huang, Sheng Di Huang and Jin Yin Hua.

Treatments for psoriasis, eczema, melanoma and other skin disorders, inflammatory and autoimmune ailments and cancer are provided herein. In certain embodiments, treatments may include combinations of certain herbs, certain herbal extracts and/or certain herbal molecular components alone or in combination with known antimetabolite, antifolate, anti-inflammatory, or autoimmune treatments and other known and/or described treatments, particularly to treat psoriasis or reduce psoriatic suffering and symptoms, or to treat eczema or blisters, redness or eczematic soreness or itching or crusty skin caused by eczema, or to treat inflammation or an inflammatory condition, or to treat melanoma or other cancer, or to reduce white cell count, tumor size or painfulness from cancer, or to treat or reduce suffering from another skin ailment, or to treat an autoimmune disease or disorder, or otherwise to treat or to reduce suffering from one or more of diseases causing or stemming from inflammation or autoimmune disease or to administer along with a known or discovered treatment to enhance effectiveness, reduce toxicity or side effects and/or to facilitate weening from a known or discovered treatment. Examples include herbal formulas including one or more of, or a combination of two or more of, Da Huang, Sheng Di Huang, and Jin Yin Hua, and/or combinations including one or more of Mu Dan Pi, Di Gu Pi, Xian He Cao, and/or Chun Gen Pi., and/or another herb, molecule or extract, or combination of herbs, molecules or extracts described herein.

IL-17/IL-23 axis plays a central role in pathology of psoriasis along with TNF-α. Recently, this arm has emerged as a new and promising therapeutic target for anti-psoriatic drugs (Lowes et al., 2013). Secretion of IL-23 from activated inflammatory and resident dendritic cells (DCs) stimulates T17 cells to secrete IL-17A and IL-17F (Zaba et al., 2009). This results in the activation of epidermis to release excessive levels of inflammatory chemokines and cytokines, which further leads to stimulation of inflammatory cascade in keratinocytes and progression of psoriasis (Bromley et al., 2008). Lately, promising therapeutic efficacy in psoriatic conditions has been demonstrated by IL12/IL23 inhibitor; Ustekinumab, the IL17A inhibitors; Secukinumab and Ixekizumab, the IL17A receptor inhibitor; Brodalumab, and the IL23 inhibitors; Guselkumab and Tildrakizumab (Mease, 2015).

Although, the defects in key signaling pathways underlying the pathology of psoriasis have been recently elucidated, specific modifications and mechanisms of the psoriatic gene expression are yet to be fully understood (Boehncke and Menter 2013). The pathogenic roles of MAP kinases (Mitogen Activated Protein Kinases) and extracellular signal-regulated kinase 1/2 (ERK1/2) in psoriasis have been recently demonstrated (Mavropoulos et al., 2013). In addition, Phosphoinositide-3 kinase/protein kinase-B/mammalian (PI3K/Akt/mTOR) pathway has also been identified as a potential therapeutic target for psoriasis (Huang et al., 2014). The PKC inhibitor AEB071 is reported to possess remarkable anti-psoriatic efficacy (Skvara et al., 2008). SKLB4771, a potent and selective Fms-like tyrosine kinase-3 (FLT3) inhibitor has exhibited beneficial effects in psoriatic models (Yan et al., 2014). In addition, Topoisomerase-II inhibitors such as Camptothecin, Bimolane, Razoxane and antibiotics such as ovobiocin and nalidixic acid have been explored as anti-psoriatic agents (Lin et al., 1999; Bohr et al., 1987).

Since ancient times, traditional herbal medicines have been effectively employed for the treatment and management of various dermatological disorders, including psoriasis (Baroni et al., 2015; Koo and Arain, 1998; Tse, 2003). Traditional Chinese Medicine (TCM) has displayed promising anti-psoriatic potential in clinical investigational studies and has been characterized by various methods of treatment, increased efficacy, low recurrence, few side-effects and flexible usage of conventional drugs (Yan et al., 2015; Zhang and Qu, 2002; Zhang et al., 2014; Parker et al., 2014). Inspite of the extensive wealth of knowledge gathered over ages in the application of TCM in psoriasis, their specific mechanism of action at cellular and molecular level is still under investigation by various researchers (Zhang and Gu, 2007). Anti-psoriatic potential of 60 Chinese medicinal herbs was evaluated in HaCaT cells by determining the inhibitory effect on proliferation (Tse et al., 2006). Similarly, the anti-proliferative and anti-inflammatory mode of action of Chinese herbal medicine (Tuhuai extract) has been elucidated in murine models (Man et al., 2008).

Da Huang (rhubarb, Radix et Rhizoma Rhei), Sheng Di Huang (Chinese Foxglove Root, Radix Rehmanniae) and Jin Yin Hua (Honeysuckle Flower, Flos Lonicerae) have been widely reported as the constituents of TCM based polyherbal formulae for effective management of psoriasis (Zhou D et al., 2014). Da huang has demonstrated anti-inflammatory, vasodilatory (Moon et al., 2006), anticancer (Huang et al., 2007) and anti-angiogenic (He et al., 2009) properties. Sheng Di Huang has been associated with anti-diabetic (Ceylan-Isik et al., 2008), anti-inflammatory and neuroprotective potential (Viljoen et al., 2012). Also, Jin Yin Hua is reported to possess antipyretic, antibiotic, antioxidant, and anti-inflammatory activities (Kang et al., 2014; Shang et al., 2011).

SIRB-001 is a novel concoction of 3 TCM based herbs in the ratio 1:1:3, namely Da Huang (Radix et Rhizoma Rhei), Sheng Di Huang (Radix Rehmanniae) and Jin Yin Hua (Flos Lonicerae). In the present study, we investigated the anti-psoriatic potential of SIRB-001 and elucidated its mechanism of action by determining its anti-proliferative, pro-apoptotic, anti-inflammatory and anti-angiogenic activity. Inhibitory effect of SIRB-001 on proliferation of keratinocytes in vitro was assessed using HaCaT cell line as the target cell population. Induction of apoptosis by SIRB-001 was studied in HaCaT cells employing a panel of early, mid and late markers. Inhibition of inflammatory cytokines (TNF-α, IFN-γ, IL-6), NO and sPLA₂ by SIRB-001 was evaluated against TNF-α triggered inflammatory conditions in HaCaT cells. Downregulation of VEGF secretion by SIRB-001 in HaCaT cells was also examined. SIRB-001 induced blockade of IL-17/IL-23 axis was determined in key immune cell populations; murine splenocytes and human monocytes (THP-1). In addition, SIRB-001 induced modulation of target markers which are integral members of signaling cascade in psoriasis were also studied. The anti-psoriatic activity of SIRB-001 was confirmed in vivo using TPA induced ear inflammation and IMQ induced psoriasis model.

Human recombinant TNF-α and cytokine-specific ELISA kits for human TNF-α, IFN-γ, IL-6, VEGF, IL-23, mouse IL-17, TNF-α, IL-6, IL-23 (Quantikine) purchased from R&D Systems, Minneapolis, Minn. and sPLA₂ estimation kit (Cayman) were used. For apoptotic assays, Nexin reagent, Mitopotential kit, Cell-cycle reagent (Guava Technologies) and Caspase-3 kit (Biovision) were used. Lipopolysaccharide (LPS, from E. coli serotype 0127:B8), Hoechst dye, Curcumin, PMA (Phorbol 12-myristate 13-acetate), MTT (3-(4,5-dimethylthiazol-2-yl)-2,5-diphenyltetrazolium bromide), L-glutamine, Sulphanilamide, NED (N-(1-naphthyl) ethylenediamine dihydrochloride), Paraformaldehyde, PEG, Triton X-100, DMEM and RPMI 1640 (Invitrogen), FBS, Penicillin-streptomycin (Hyclone), Methotrexate, Phorbol 12-myristate 13-acetate (TPA), from Sigma, DMSO (Merck), Imiquad Cream (Glenmark generics, India) and Betamethasone 17-valerate (Betnovate® Skin cream, GSK) were used in the study.

For isolation of splenocytes in cell based assays, specific pathogen-free male C57BL/6 mice (20 to 30 g, 8-10 weeks) were used. Male C57BL/6 mice (20 to 30 g, 7-9 weeks) were used in TPA induced psoriasis model. Female Balb/c mice (18-30 g, 4-6 weeks) were used in IMQ induced psoriasis model. Animals were obtained from National Centre for Laboratory Animal Sciences (NCLAS), National Institute of Nutrition (NIN), Hyderabad, India. Mice were kept in the in-house animal facility maintained at 22±3° C. and 55±15% relative humidity with 12 hr light-dark cycle. They were given autoclaved pelleted feed and filtered drinking water ad libitum. All experiments employing the mice were performed under the protocols approved by the Institutional Animal Ethics Committee (IAEC) of Dabur Research Foundation.

### Preparation of SIRB-001

Da huang roots were supplied by Spring Wind herbs Inc., Berkeley Calif. Sheng di huang roots and Jin yin hua flowers were supplied by Phoenix Medical Limited, UK. To prepare SIRB-001 extract, three herbs were mixed in the ratio of 1:1:3 (Da Huang: Sheng Di Huang: Jin Yin Hua). Herbs were ground in the mixer for 2-3 min to obtain a fine powder. 25 g of powder was weighed and diluted with distilled water in a ratio of 1:20 (g of herbs: ml of water). After soaking the herbs for 15-20 min, the mixture was boiled to 85-90° C. The temperature was allowed to cool down and subsequently, the mixture was cooked at 70° C. for a total of 1 h. The mixture was strained and the extract obtained after filtration was centrifuged at 5000 rpm for 15 min. The supernatant was filter sterilized by passing through 0.2 μm Millipore® syringe filter. The clear filtrate was stored at 4° C. and used as the main stock. The yield of SIRB-001 extract was 30 mg/ml. For lyophilization, 100 ml of SIRB-001 extract was transferred to the lyophilization vessel and lyophilized for 24-40 h at −75° C.±5° C. and vacuum ≤100 millitorr.

### Fingerprint HPLC Analysis of Herbs

The experiment was performed using a high performance liquid chromatography (HPLC-Shimadzuschromatograph-LC-2010CHT) equipped with a quaternary solvent delivery system, an autosampler and a photodiode array detector (DAD). Fingerprint analysis of SIRB-001 was carried out under gradient conditions using C18 column (4.6 mm 250 mm) packed with 5 μm diameter particles. The mobile phase consisted of 10 mM ammonium acetate containing 0.1% acetic acid (A) and acetonitrile: methanol 1:1 (B). The composition gradient was: 90% of A (0-8 min); 90-80% of A (8-35 min); 80% of A (35-45 min); 80-75% of A (45-60 min); 75% of A (60-70 min); 75-70% (70-75 min); 70% (75-85 min). After 85 mins the system was returned to its original condition (mobile phase B at 90%) in 90 mins and equilibrated for 10 mins before next injection. The flow rate was 1.0 ml/min with injection volume 10 μl. The column oven was set at 30° C. and the eluent was monitored at 220 nm. All the samples and mobile phase were filtered through 0.45 μm membrane filter (Millipore®) and then degassed by ultrasonic bath prior to use. The retention time and UV spectra of major peaks were analyzed. The presence of 3 marker compounds was investigated, namely, chlorogenic acid, Acteoside and Rhein. Identification of these compounds was performed by comparing their retention time and UV absorption spectrum with commercial standards.

### Anti-Proliferative Assay

HaCaT (Human epidermal keratinocyte cell line) was obtained from NCCS (National Centre for Cell Science, Pune, India) and maintained in Dulbecco's modified Eagle's medium (DMEM) with 10% FBS, 100 U/ml penicillin and 10 μg/ml streptomycin. Cells were maintained in a humidified atmosphere with 5% CO₂ at 37° C. and trypsinized on attaining 70-80% confluence. Cells were used within passage number 10. SIRB-001 was diluted in serum free medium (SFM) to achieve final dilutions for treatment ranging from 1: 100-1:5 (v/v). Cells were seeded in 96-well culture plates at a density of 10,000 cells/well in DMEM+10% FBS. After 24 h, cells were treated with SIRB-001 at dilutions ranging from 1:100-1:5 (v/v). The cytotoxic/anti-proliferative effects on HaCaT cells were determined after 48 h of incubation by addition of MTT (0.5 mg/ml). For assessment of cell viability, cells were treated with SIRB-001 for 24 h. Curcumin (0.1 μM-100 μM) was included as a positive control (PC). After 3 h, the suspension was removed and cells were resuspended in 150 μl of DMSO to dissolve the formazan complex. Absorbance of samples was measured at 540 nm. Inhibition of cellular proliferation mediated by SIRB-001 was determined as:

Inhibition of proliferation (% of control)={(Y−X)/Y}*100, Where, X=Absorbance of cells treated with SIRB-001 at 540 nm; Y=Absorbance of control cells (medium treated) at 540 nm. The morphological changes in HaCaT cells treated with SIRB-001 were observed using the inverted microscope and photo-documented (at 100×).

### Apoptosis Assays

HaCaT Cells were seeded in the growth medium at following densities for various apoptosis markers:

Annexin-V, Mitopotential & Cell cycle analysis—0.5×10⁶ cells in 6-well culture plate, Casapase-3-1.5×10⁶ cells in 6-well culture plate, DNA fragmentation—1×10⁶ cells in 90-mm culture petridish. After 24 h of incubation, cells were treated with SIRB-001 at dilutions ranging from 1:10-1:5 (v/v). Curcumin (20 μM) was used as a positive control.

### Externalization of Phosphatidyl Serine (PS) by Annexin-V Staining

After 24 h of treatment, cells were harvested and centrifuged at 300 g for 5 min. Cell pellet was resuspended in Phosphate Buffer Saline (PBS). 100 μl of cell suspension was incubated with 100 μl of Nexin reagent (Annexin V/7-AAD) for 20 min at RT in dark. Samples were acquired on a flow cytometer (Guava technologies) to determine percent population of cells in 4 quadrants viz., viable, early apoptotic, late apoptotic and necrotic cells.

### Mitochondrial Membrane Potential (JC-1 Staining)

After 24 h of incubation, cells were harvested and centrifuged at 300 g for 5 min. Cell pellet was resuspended in 200 μl of Phosphate Buffer Saline (PBS). 4 μl of 50× cell staining solution (JC-1 and 7-AAD) was added to each tube and mixed well. Samples were incubated for 30 min at 37° C. in CO₂ incubator and acquired on flow cytometer (Guava technologies) to detect early vs. late apoptotic cells.

### Caspase-3

After 24 h of incubation, cells were washed with PBS. Cells were lysed using 1× cell lysis buffer over ice for 10 min. Cells were centrifuged at 10,000 g for 1 min and supernatant was collected. Casapse-3 activation was determined by incubating supernatants with reaction mixture containing p-NA conjugated DEVD (Asp-Glu-Val-Asp) as substrate at 37° C. for 2 h. Absorbance was determined at 405 nm.

### Cell Cycle

Cells were harvested after 48 h of incubation and centrifuged at 450 g for 5 min. Cells were washed in PBS and fixed in ice cold 70% ethanol for 24 h at 4° C. After washing with PBS, cell pellet was resuspended in 200 μl of cell cycle reagent (Propidium Iodide) and incubated at RT for 30 min in dark. Percentage of pro-apoptotic cells in Sub(G0/G1) phase was determined by acquiring the cells on flow cytometer (Guava technologies).

### DNA Fragmentation

After 48 h of treatment, cells were stained using Hoesct dye. For quantitative estimation of DNA fragmentation, cells were centrifuged at 800 rpm for 3 min. Cell pellet was resuspended in 80 μl of DNA lysis buffer (0.1% Triton X-100, 5 mM Tris-HCl, pH 8.0, 20 mM EDTA) and subsequently PEG (2.5%) and NaCl (1 M) were added. After incubation on ice for 10 min, samples were centrifuged at 16000 g for 10 min. Supernatant was collected, 0.2 mg/ml of Hoechst dye was added to each tube and incubated at RT for 20 min. The fluorescence was measured at excitation and emission wavelengths of 360 nm and 460 nm respectively.

For visualization of DNA fragmentation, cells were fixed in 150 μl of 4% paraformaldehyde for 10 min. After fixation, cells were centrifuged at 3000 rpm for 3 min. Cells were washed with ice-cold PBS and incubated with 5 μg/ml of Hoechst dye at 37° C. in dark for 5 min. Stained cells were observed under fluorescent microscope for visualization of apoptotic cell populations.

### Estimation of Pro-Inflammatory Markers (TNF-α, IFN-γ, IL-6, sPLA₂ and NO) in HaCaT Cells

To determine the inhibitory effect of SIRB-001 on cytokines secreted by keratinocytes, HaCaT cells were seeded (1×10⁶ cells/well in 6-well culture plates) in DMEM with 10% FBS and incubated for 24 h. Cells were sera starved (DMEM+0.1% FBS) for another 24 h and treated with SIRB-001 at dilutions ranging from 1:10-1:5 (v/v) in the presence of inflammatory stimulus human-TNF-α (10 ng/ml). Curcumin (10 and 20 μM) was included as a positive control (PC). Cells treated with TNF-α alone were included as control. After 24 h of incubation, cell-free supernatants were analyzed for secreted levels of TNF-α, IFN-γ, IL-6, sPLA₂ by ELISA. To determine NO levels, 100 μl of culture supernatant was mixed with 100 μl of Griess reagent (equal volumes of 0.1% NED (N-(1-naphthyl) ethylenediamine dihydrochloride) and 1% sulfanilamide in 5% phosphoric acid) and incubated for 30 min at room temperature.

Percentage inhibition of secreted pro-inflammatory markers in culture supernatants was determined as:

{(B−A)/B}*100, Where A=Concentration of marker secreted by TNF-α stimulated cells treated with SIRB-001, B=Concentration of marker secreted by TNF-α alone stimulated cells

### VEGF Inhibition in HaCaT Cells

HaCaT cells were plated at density of 1×10⁶ cells/well in 6-well culture plates in DMEM+10% FBS and incubated for 24 h. After sera starvation for another 24 h, cells were treated with SIRB-001 at dilutions ranging from 1:100-1:8 (v/v). Curcumin (10 and 20 μM) was included as a positive control (PC). Untreated cells served as control. After 24 h of incubation, cell-free supernatants were analyzed for secreted levels of VEGF by ELISA. Inhibition of VEGF secretion was calculated with respect to untreated (basal) levels.

### Determination of IL-17/IL-23 Secretion in Immune Cells

Murine splenocytes suspension was prepared by aseptically removing the spleens from C57BL/6 mice. Spleens were homogenized and the red blood cells were lysed by treatment with RBC lysis buffer. After washing with PBS, 0.2×10⁶ splenocytes were seeded/well in 96-well plates and treated with SIRB-001 at 1:100-1:5 (v/v). After 24 h, non-cytotoxic concentrations were determined by MTT assay. For IL-17 estimation, 1×10⁶ splenocytes/well were treated with SIRB-001 at dilutions ranging from 1:100-1:5 (v/v), along with the inflammatory stimulation; Con-A (5 μg/ml) in 24-well plates. Cells treated with Con-A alone served as control. Curcumin (10 and 20 μM) was included as a positive control. After 24 h of incubation, supernatants were collected and levels of IL-17 were estimated by ELISA.

For IL-23 estimation, the human monocytic cell line; THP-1 was obtained from ATCC (American Type Culture Collection, Manassas, Va., USA). Cells were grown in DMEM+10% FBS, 1% nonessential amino acids, 1% glutamine, 100 U/ml penicillin, and 10 μg/ml streptomycin. 30,000 THP-1 cells were treated with SIRB-001 at dilutions ranging from 1:100-1:5 (v/v) in 96-well plates. Non-cytotoxic concentrations were determined by MTT assay after 24 h. For IL-23 assay, 1×10⁶ THP-1 cells were treated with SIRB-001 at dilutions ranging from 1:100-1:5 (v/v), along with inflammatory stimulus LPS (1 μg/ml)+IFN-γ (100 ng/ml) in 6-well plates. Cells treated with LPS+IFN-γ alone served as control. Curcumin (10 and 20 μM) was included as a positive control. Supernatants were collected from each well after 24 h and levels of IL-23 were estimated by ELISA.

### Signaling Markers

Activity of various kinases was evaluated in biochemical cell-free systems using purified kinases. For AKT1 (PKB alpha), FLT3, MAPK1 (ERK2) and MAP2K1 (MEK1), the assay mixture consisted of purified enzymes and fluorophore conjugated-substrates in 50 mM HEPES pH 7.5, 0.01% BRIJ-35, 10 mM MgCl₂, 1 mM EGTA and PRKCA (PKC alpha) in 50 mM HEPES pH 7.5, 0.01% BRIJ-35, 10 mM MgCl₂, 500 μM EGTA, 2 mM CaCl₂, 1× Novel Lipid Mix, 0.01% NaN₃. The reaction was developed after 1 h of incubation and fluorescence was read to determine kinase inhibition. NF_{K}B (TNF-alpha) activity and DNA damage/p53 response was determined in cell lines over expressing the particular pathways system (NFₖB-bla THP-1 and p53RE-bla HCT-116 cells line).

Topoisomerase-II activity was evaluated in cell free system using purified enzyme. KDNA was treated with SIRB-001 at v/v dilutions (1:10, 1:20 and 1:30) in the presence of Topoisomerase II enzyme (10 U). Reaction mixture was incubated at 37° C. for 30 min. Sample was analyzed on agarose gel electrophoresis and images were captured using gel documentation system (Biorad).

### In Vivo TPA Induced Ear Inflammation Model

Anti-inflammatory potential of SIRB-001 was assessed in vivo in TPA (Phorbol 12-myristate 13-acetate) induced ear-inflammation model in male C57BL/6 mice (7-9 weeks, n=7/group) (Man et al., 2008). 5 mg/ml stock of TPA was prepared in DMSO and diluted to 1:50 with methanol to obtain final TPA concentration of 100 μg/ml. On day 0, 2, 4, 7 and 9, TPA was applied topically on both right and left ear (10 μl each on ventral and dorsal side) of mice. SIRB-001 was administered to mice orally (500 mg/kg) and topically (1:4 in 2% DMSO+98% methanol) for 10 days. Methotrexate (MTX) was included as positive control at dose of 10 mg/kg (oral) in 0.25% Na-CMC. For topical application, betamethasone-17-valerate was used as positive control at concentration of 0.01%. For comparison, mice treated with TPA+0.25% Na-CMC and TPA+2% DMSO+98% methanol were included as control group for oral and topical treatment group respectively. Negative controls for oral and topical groups included 0.25% Na-CMC alone and 2% DMSO+98% methanol respectively. The ear thickness was measured daily using digital caliper. On day 10, ear punch biopsy from right ear (4 mm) was collected for histopathological analysis. Remaining ears were stored at −80° C. for quantification of inflammatory cytokines (TNF-α, IL-6) in the homogenates using ELISA.

### IMQ Induced Psoriasis Model

Anti-psoriatic potential of SIRB-001 was assessed in vivo in IMQ (Imquimod) induced psoriasis model in female Balb/c mice (n=5/group) (Van der Fits et al., 2009). 63 mg of IMQ cream (5%) (containing 3.125 mg of IMQ) was applied topically each on dorsal area (shaved back) and right ear of mice for 8 days. SIRB-001 was administered orally at dose of 500 mg/kg, 1000 mg/kg and 1500 mg/kg. Methotrexate (MTX) was included as positive control at dose of 10 mg/kg (oral) in 0.25% Na-CMC. Test items and reference compounds were administered for 8 days. Ear thickness and dorsal skin thickness was measured daily using digital caliper. On day 9, 4 mm ear punch biopsy was collected for histopathological analysis (H & E staining). Remaining ear was excised and stored at −80° C. for analysis of inflammatory cytokines (IL-23/IL-17) analysis.

### Statistical Analysis

For in vitro studies, experimental values were expressed as arithmetic mean±SEM. Statistical differences between control and treatment groups were determined using One-way Anova with Dunnett's multiple comparison post test. For animal studies, data were expressed in Mean±S.E.M and analyzed by Two way Anova and One-way Anova followed by Posthoc Bonferroni and Dunett's test respectively for statistical significance.

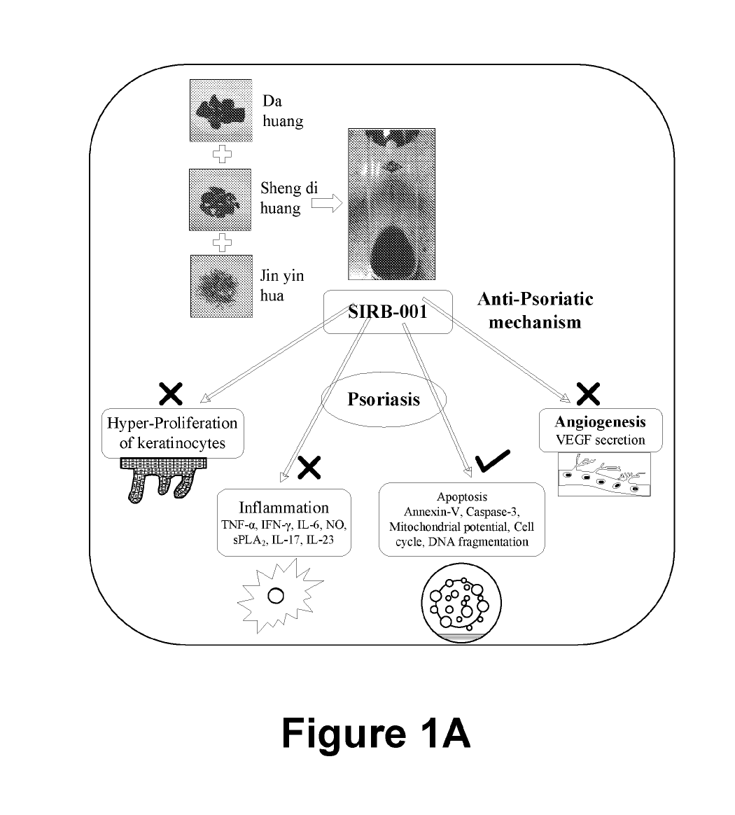

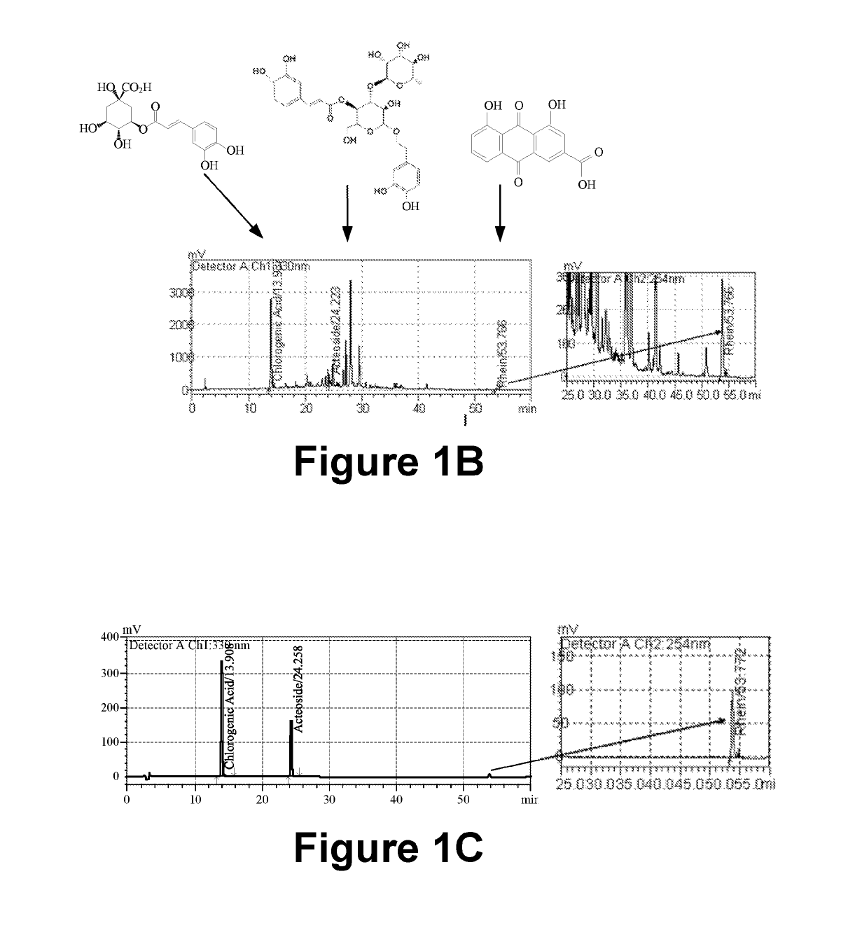

FIG. 1B illustrates a representative HPLC profile of SIRB-001, detection UV was at 230 nm and 330 nm. Chromatographic conditions are described in section 2.4 of materials and methods.

FIG. 1C illustrates a representative HPLC profile of standard marker compounds.

### HPLC Analysis of SIRB-001

HPLC fingerprinting of SIRB-001 revealed the presence of the chlorogenic acid (tR=13.98 min), Acteoside (tR=24.22 min) and Rhein (tR=53.76 min) (FIGS. 1A-1B). They were identified by comparisons to the retention times and UV spectra of authentic standards analyzed under identical analytical conditions.

### SIRB-001 Induced Inhibitory Effect on Proliferation of HaCaT Cells

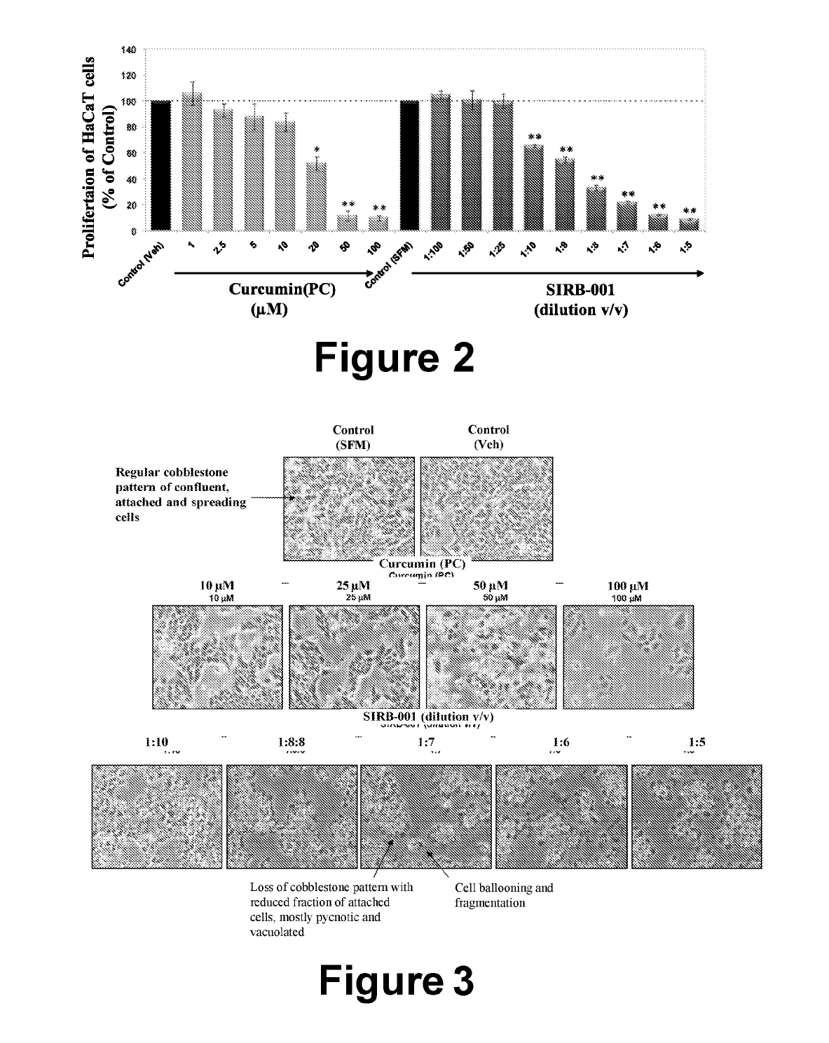

Anti-proliferative potential of SIRB-001 was determined in keratinocytes (HaCaT cells) after 48 h of treatment by MTT assay. At dilutions 1:100-1:25 (v/v), SIRB-001 did not result in any loss of cell viability. However, in the dilution range of 1:10-1:5, SIRB-001 demonstrated significant inhibitory effect (p<0.01) on the proliferation of HaCaT cells as compared to control (untreated) cells in a dose-dependent manner (FIG. 2). IC₅₀ value of SIRB-001 for inhibition of HaCaT cells proliferation of was determined as 1:8.8 (v/v). Curcumin (PC) also resulted in significant inhibition (p<0.05, p<0.01) of proliferation of keratinocytes (HaCaT cells) with an IC₅₀ of 20.7 μM.

FIG. 2 illustrates an anti-proliferative effect of SIRB-001 in keratinocytes (HaCaT cells) after 48 h of treatment. 10,000 cells/well were treated with SIRB-001. Proliferation was determined by MTT assay and expressed as a percentage (Mean±S.E.M.) of control cells. n=3. *: p<0.05, **: p<0.01, significantly different from the control group.

### SIRB-001 Exerted Alterations in Morphology of HaCaT Cells

HaCaT cells were treated with various dilutions of SIRB-001 (1:10-1:5; including IC₅₀) for 48 h and observed under the phase-contrast microscope for any changes in cell-morphology. Treatment with SIRB-001 led to noticeable alterations in the typical morphology of HaCaT cells (FIG. 3). Control (untreated) HaCaT cells displayed a regular cobblestone pattern, which is a characteristic feature of HaCaT cells. Early confluent, packed, attached and spreading cells with clear nuclear areas including prominent, dark nucleoli were observed. However, treatment of HaCaT cells with SIRB-001 revealed cytotoxic effects with inhibition of proliferation, as indicated by a reduced density of attached cells (mostly pycnotic and vacuolated), loss of regular cobblestone pattern and widening of the intracellular spaces with detachment of cells. Additionally, striking drug-induced alterations of cell-shape (impeded cell spreading, ballooning and fragmentation) were also noticed. Similar changes in cellular morphology were also observed with Curcumin (PC) at 20 μM.

FIG. 3 illustrates alterations in the morphology of keratinocytes (HaCaT cells) induced by SIRB-001 after 48 h of treatment. Control (untreated) cells demonstrate a typical cobblestone pattern, with early confluent, attached and spreading cells. Detachment of cells was observed in SIRB-001 (1:10-1:5 v/v) treated cells with ballooning and fragmentation. (100× magnification).

### SIRB-001 Induced Activation of Apoptotic Markers

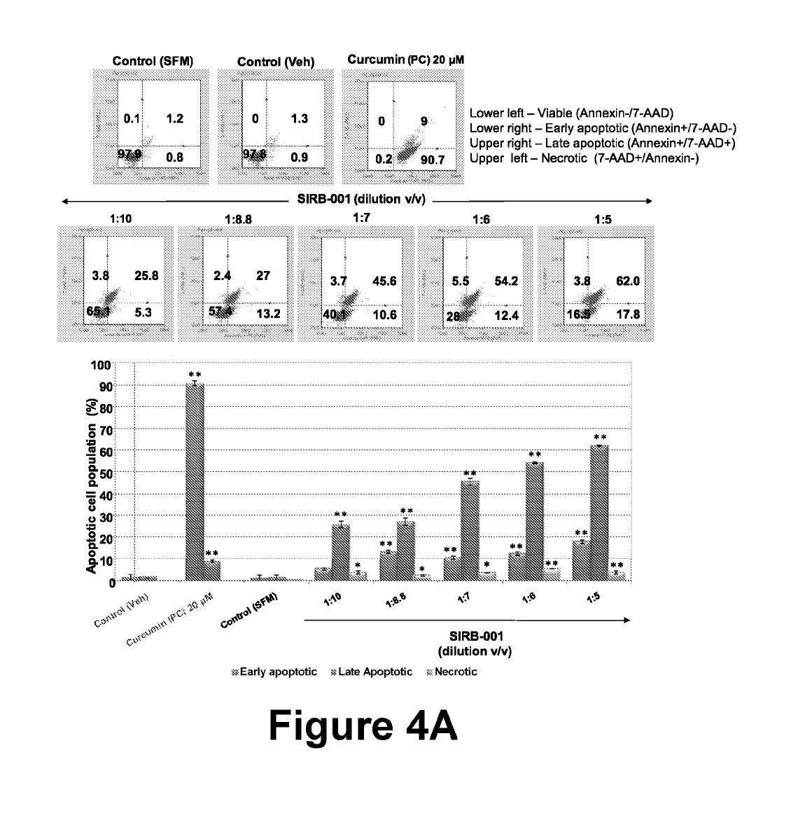

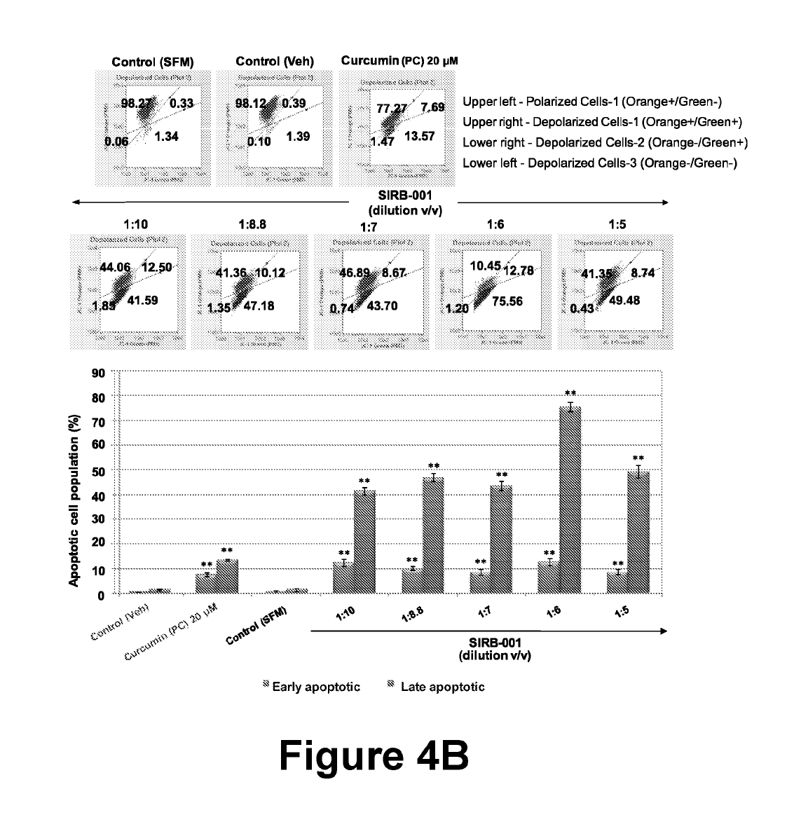

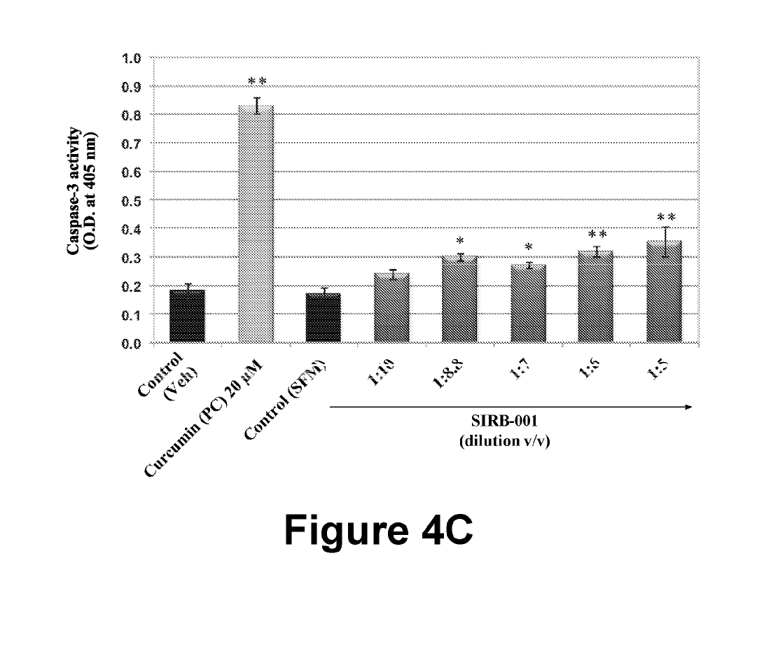

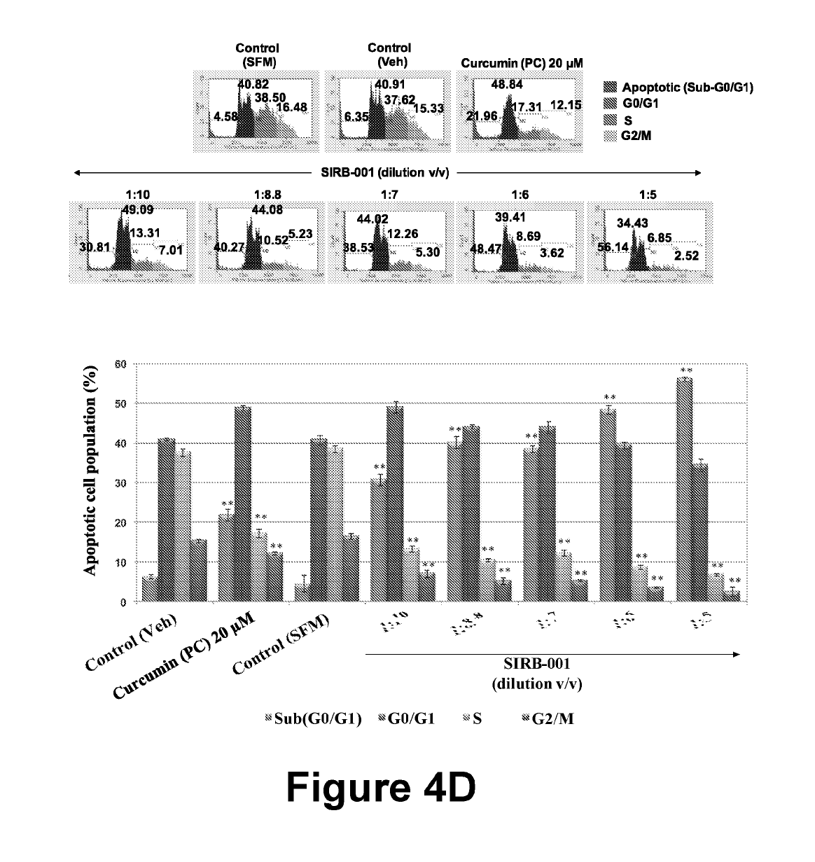

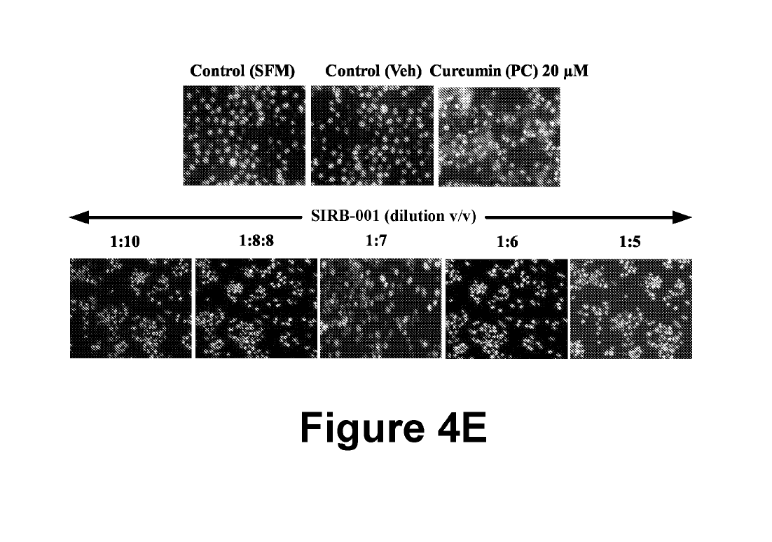

Anti-proliferative potential of SIRB-001 was found to be mediated via induction of apoptosis. HaCaT cells were treated with SIRB-001 at effective concentrations (1:10-1:5 v/v) to study resultant effects on apoptosis. FIGS. 4A-4E represents activation of apoptotic markers by SIRB-001.

### PS Externalization by Annexin-V Assay

The apoptotic potential of SIRB-001 mediated by translocation of Phosphatidyl Serine (PS) from the inner to the outer leaflet of the plasma membrane of HaCaT cells was evaluated after 24 h of treatment by staining with Annexin-V/7-AAD reagent and analyzed using flow cytometry. At dilutions; 1:10-1:5 (v/v), SIRB-001 exhibited significant (p<0.01) pro-apoptotic activity by induction of PS externalization on cell membrane and increase in the percentage population of early (Annexin+/7-AAD−) and late apoptotic (Annexin+/7-AAD+) cells as compared to control (untreated) cells (FIG. 4A). At 1:5, SIRB-001 induced significant increase (p<0.01) in the percentage of early and late apoptotic cell populations by a maximum of 22.3 & 51.7 fold respectively with respect to control cells. There was an increase in necrotic population also. Curcumin at 20 μM (PC) also induced significant (p<0.01) increase in early and late apoptotic cell populations by 100.8 and 6.9 fold respectively.

### Mitochondrial Depolarization

The apoptotic effect of SIRB-001 via the loss of mitochondrial transmembrane potential (ΔΨ) in HaCaT cells was evaluated after 24 h of treatment by incubating treated cells with Mitopotential reagent (JC-1/7-AAD). Resultant modulation in non-apoptotic/polarized cells (JC-1 Orange(+)/Green(−)), apoptotic/depolarized cells-1, (JC-1 Orange (+)/Green(+)), apoptotic/depolarized cells-2 (JC-1 Orange(−)/Green (+)) was assessed by flow cytometry. Treatment of HaCaT cells with SIRB-001 (1:10-1:5 v/v) induced significant (p<0.01) loss of mitochondrial potential and led to an increase in the percentage population of apoptotic (depolarized cells) as compared to control untreated cells (polarized cells) (FIG. 4B). A maximum of 38.7 and 56.4 fold increase (p<0.01) was observed in early and late depolarized/apoptotic cell populations respectively at 1:6 dilution of SIRB-001 as compared to control cells. Curcumin at 20 μM (PC) also induced significant (p<0.01) increase in early and late apoptotic cell populations by 19.7 and 9.8 fold respectively.

### Caspase-3 Activation

Activation of Caspase-3 induced by SIRB-001 was measured after 24 h of treatment by DEVD based colorimetric assay. SIRB-001 was able to induce significant (p<0.05 and p<0.01) activation of caspase-3, when compared with control (untreated) cells (FIG. 4C). A maximum of 2.1 fold increase in Caspase-3 activation was demonstrated at 1:5 dilution of SIRB-001. Curcumin at 20 μM (PC) significantly (p<0.01) stimulated caspase-3 activity by 4.6 fold.

### Cell Cycle Distribution

Apoptotic effect of SIRB-001 was assessed via modulation of cell cycle distribution by PI staining and determining the resultant increase in percentage of pro-apoptotic cell population (Sub-G0/G1) after 48 h of treatment by flow cytometric analysis. A significant (p<0.01) increase in the percentage of pro-apoptotic population was induced by SIRB-001 as compared to control cells, reaching a maximum of 12.3 fold at 1:5 dilution of SIRB-001 (FIG. 4D). In addition, significant (p<0.01) decrease was also induced by SIRB-001 in S-phase and G2/M phase of cell cycle with a maximum of 5.6 fold and 6.5 fold inhibition respectively at 1:5 dilution. Curcumin at 20 μM (PC) displayed significant (p<0.01) increase in cell populations corresponding to pro-apoptotic phase (Sub-G0/G1) by 3.5 fold along-with a decline in S-phase and G2/M phase of cell cycle by 2.2 and 1.3 fold respectively.

### DNA Fragmentation

Apoptotic effect of SIRB-001 by increase in DNA fragmentation was determined after 48 h by staining the treated cells with Hoechst dye and subsequently analyzing for DNA fragmentation in qualitative and quantitative assays. A significant increase (p<0.01) in DNA fragmentation was observed with a maximum of 1.8 fold (78%) at 1:6 and 1:5 dilutions of SIRB-001 as compared to control cells (FIG. 4E). Curcumin at 20 μM (PC) resulted in significant (p<0.0) increase in DNA fragmentation by 1.6 fold.

Photomicrographs of Hoechst dye stained cells were obtained using fluorescence microscope and the effects of SIRB-001 on DNA fragmentation were examined. Untreated cells stained with Hoechst dye showed a pale blue color and nuclei were conspicuous with a dark blue color (control cells). However cells treated with SIRB-001 and positive control Curcumin (20 μM) exhibited a rough cellular outline with brighter and more concentrated DNA chromatin.

FIG. 4A illustrates SIRB-001 induced externalization of PS on HaCaT cell membrane by Annexin-V/7-AAD staining after 24 h of treatment. 0.5×10⁶ cells/well were treated with SIRB-001 and apoptosis was analyzed using Annexin V/7-AAD dual staining. Representative results demonstrate percentage population of early apoptotic (Annexin+/7-AAD−), late apoptotic (Annexin+/7-AAD+) and necrotic (Annexin+/7-AAD+) cells (n=3). Total 10,000 events were acquired. *: p<0.05, **: p<0.01, significantly different from the control group.

FIG. 4B illustrates depolarization of mitochondrial membrane potential in HaCaT cells by SIRB-001 detected by JC-1/7-AAD staining after 24 h of treatment. 0.5×10⁶ cells/well were treated with SIRB-001 and apoptosis was analyzed using JC-1/7-AAD dual staining. Representative results demonstrate percentage population of viable/polarized cells (JC-1 Orange(+)/Green(−)), apoptotic/depolarized cells-1, (JC-1 Orange (+)/Green(+)), apoptotic/depolarized cells-2 (JC-1 Orange(−)/Green (+)) (n=3). Total 10,000 events were acquired. **: p<0.01, significantly different from the control group.

FIG. 4C illustrates caspase-3 activation by SIRB-001 in HaCaT cells after 24 h of treatment. 1.5×10⁶ cells/well were treated with SIRB-001 and induction of Caspse-3 activity was assessed in cells by DEVD based colorimetric method. Data represents Mean±S.E.M. of experiments (n=3). *: p<0.05, **: p<0.01, significantly different from the control group.

FIG. 4D illustrates apoptotic effect of SIRB-001 via modulation of cell cycle distribution in HaCaT cells by PI staining after 48 h of treatment. 0.5×10⁶ cells/well were treated with SIRB-001 and apoptosis was detected by PI staining method. Representative results demonstrate percentage population of cells in various phases of cell cycle; pro-apoptotic (sub (G0/G1), G0/G1, S and G2/M (n=3). *: p<0.05, **: p<0.01, significantly different from the control group.

FIG. 4E illustrates SIRB-001 induced enhancement of DNA fragmentation in HaCaT cells by Hoechst staining after 48 h of treatment. 1×10⁶ cells/well were treated with SIRB-001 and apoptosis was detected by Hoechst staining method. Results represent Mean±S.E.M. of experiments (n=3). **: p<0.01, significantly different from the control group. Photomicrographs represent changes in cell morphology under fluorescence microscope.

### Inhibition of Inflammatory Markers in HaCaT Cells by SIRB-001

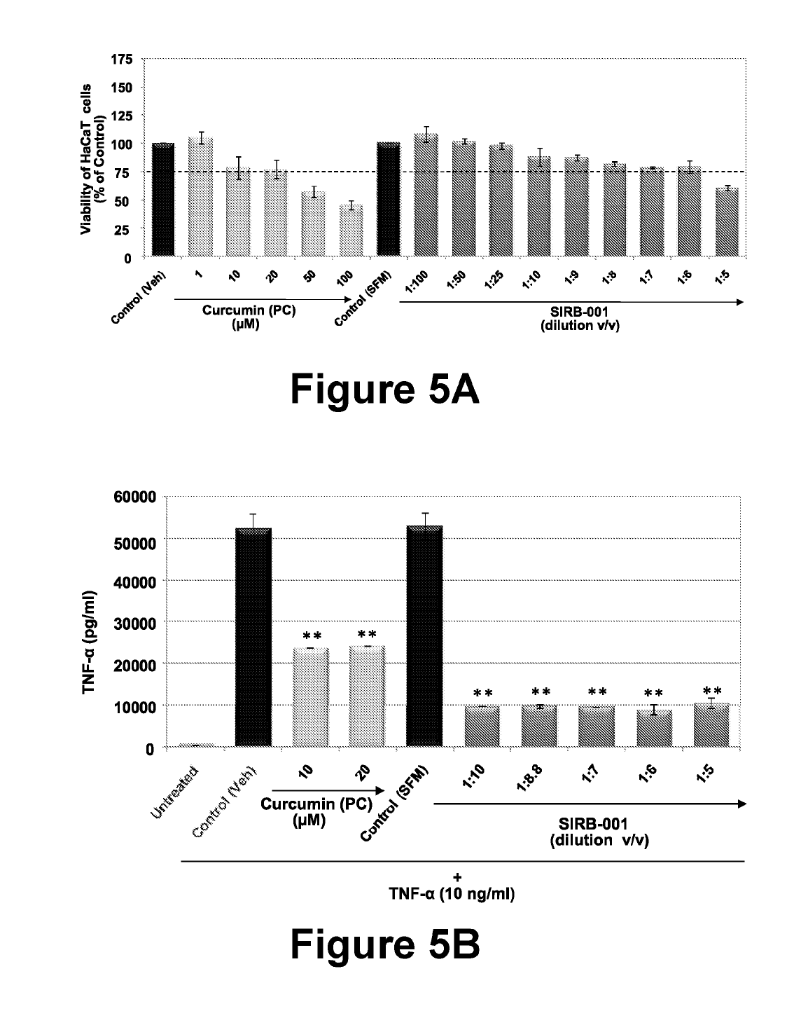

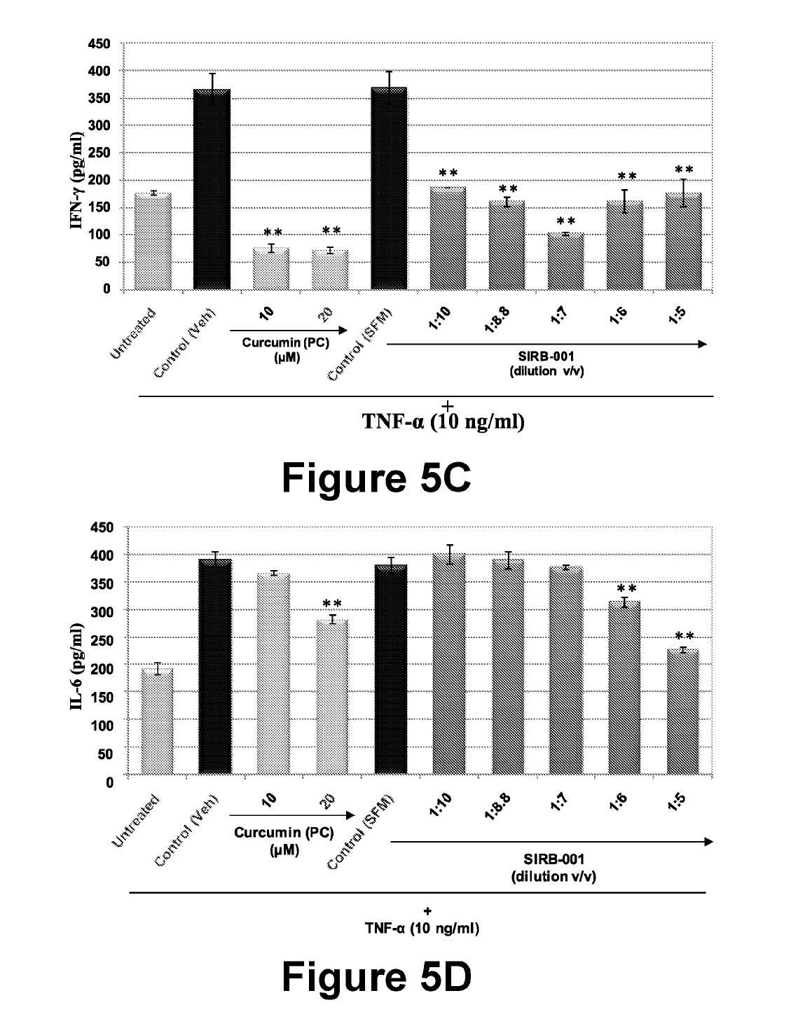

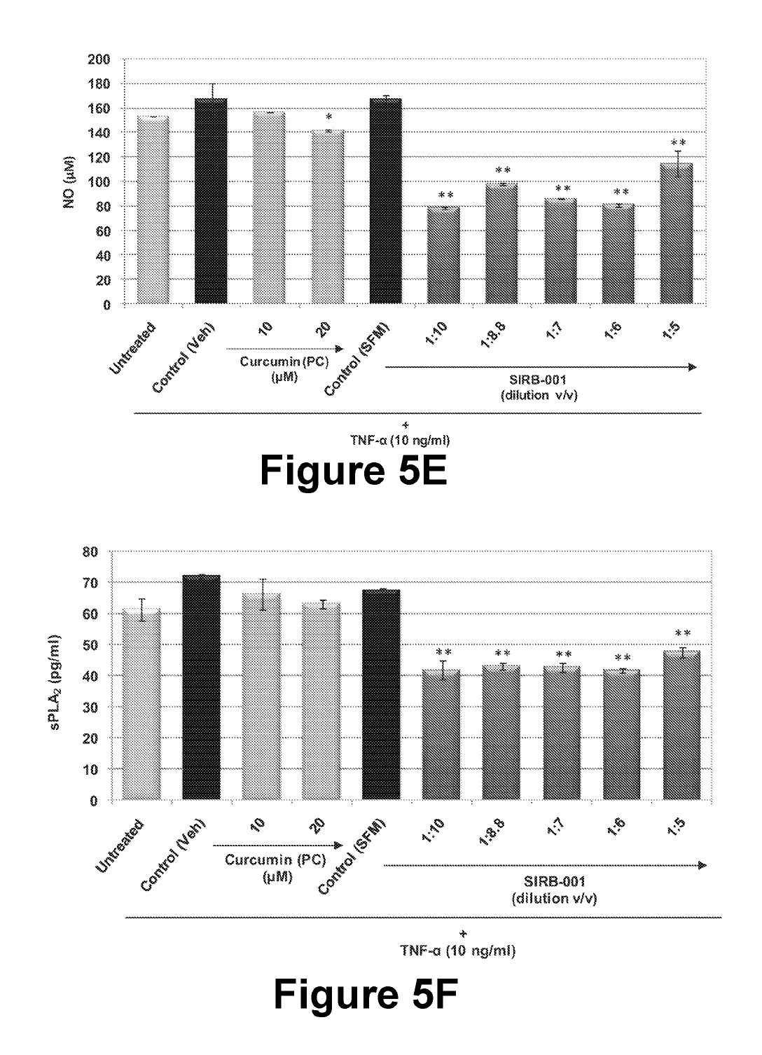

FIG. 5A illustrates the effect of SIRB-001 on viability of HaCaT cells after 24 h of treatment. Treatment of HaCaT cells with SIRB-001 at dilutions ranging from 1:100-1:6 resulted in >75% cell viability as compared with control (100%). However at 1:5 dilution, cell viability was reduced to 40% of the control. The inhibitory effect of SIRB-001 on the secretion of key pro-inflammatory markers (which are crucial in psoriasis) was assessed at dilutions ranging from 1:10-1:5 (effective concentration range for HaCaT proliferation at 48 h) in presence of inflammatory stimulus TNF-α (10 ng/ml) after 24 h of treatment (FIGS. 5B-5F).

### TNF-α

The secretion of TNF-α was enhanced from 475 pg/ml in untreated HaCaT cells (basal levels) to 52.8 ng/ml in TNF-α (10 ng/ml) stimulated cells after 24 h. Treatment of HaCaT cells with SIRB-001 (1:10-1:5 v/v) significantly (p<0.01) downregulated TNF-α secretion against TNF-α stimulated levels at all the dilutions tested (FIG. 5B). Curcumin (PC) at 10 μM and 20 μM was also able to significantly (p<0.01) inhibit TNF-α secretion.

### IFN-γ

Stimulation of HaCaT cells with TNF-α (10 ng/ml) resulted in increased secretion of IFN-γ from 176.5 pg/ml (untreated cells) to 370 pg/ml (TNF-α stimulated cells). SIRB-001 exhibited significant (p<0.01) inhibition of IFN-γ at all the dilutions tested (1:10-1:5 v/v) (FIG. 5C). Curcumin (PC) at 10 μM and 20 μM demonstrated significant (p<0.01) downregulation of IFN-γ production.

### IL-6

Secretion of IL-6 was enhanced from 192.5 pg/ml in untreated cells to 380.7 pg/ml in TNF-α stimulated cells. This stimulated secretion of IL-6 was downregulated by SIRB-001 significantly (p<0.01) at 1:6 and 1:5 v/v dilutions (FIG. 5D). Curcumin (PC) at 20 μM resulted in significant (p<0.01) inhibition of IL-6 against TNF-α stimulated levels.

### NO and sPLA₂

SIRB-001 (1:10-1:5) demonstrated significant (p<0.01) downregulation of NO and sPLA₂ production from TNF-α stimulated levels in HaCaT cells after 24 h of treatment (FIGS. 5E-5F). Curcumin (PC) at 20 μM inhibited NO and sPLA₂ secretion from stimulated levels.

FIGS. 5A-5F illustrate SIRB-001 induced inhibition of inflammatory markers in HaCaT cells after 24 h of treatment. (5a) Viability of HaCaT cells (10,000 cells/well) after 24 h of treatment with SIRB-001. Cell viability was determined by MTT assay and expressed as a percentage (Mean±S.E.M.) of control cells (n=3). HaCaT cells (1×10⁶ cells/well) were co-treated with SIRB-001 (1:10-1:5) and inflammatory stimulus TNF-α (10 ng/ml). Inhibitory effect of SIRB-001 on TNF-α induced secretion of (5b) TNF-α, (5c) IFN-γ, (5d) IL-6, (5e) Nitric Oxide (NO), and (5f) sPLA₂ in culture supernatants by ELISA. Results are expressed as (Mean±S.E.M. n=3), *: p<0.05, **: p<0.01, significantly different from the TNF-α treated control group.

### SIRB-001 Treatment Down Regulated VEGF Secretion in HaCaT Cells

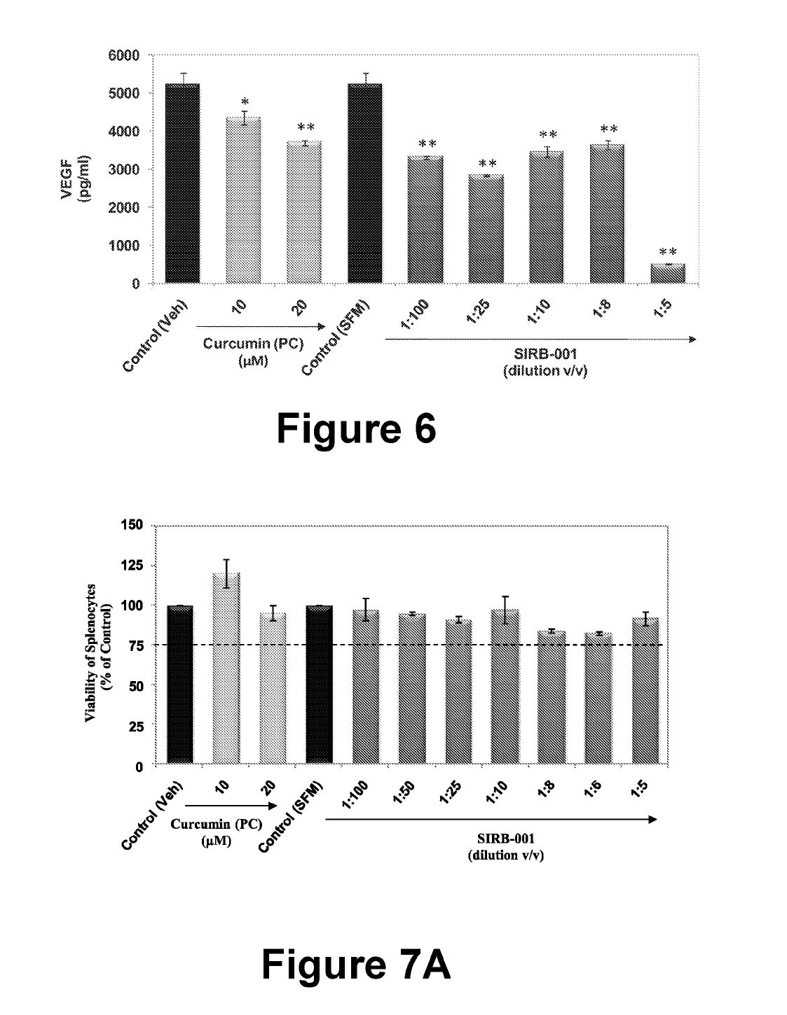

The ability of SIRB-001 to target angiogenic arm by down-regulating the secretion of VEGF from HaCaT cells was measured after 24 of treatment. From basal (untreated) levels of 5.2 ng/ml in untreated cells, secretion of VEGF was significantly (p<0.01) inhibited by SIRB-001 at dilutions 1:100-1:5 (FIG. 6). Curcumin (PC) at 10 μM and 20 μM resulted in significant (p<0.05, p<0.01) inhibition of VEGF secretion.

FIG. 6 illustrates downregulation of VEGF secretion by SIRB-001 in HaCaT cells after 24 h of treatment. HaCaT cells (1×10⁶ cells/well) were treated with SIRB-001 and VEGF secretion in culture supernatants was determined by ELISA. Results are expressed as (Mean±S.E.M. n=3), *: p<0.05, **: p<0.01, significantly different from the TNF-α-treated control group.

### SIRB-001 Exhibits Inhibition of IL-17/IL-23 Axis

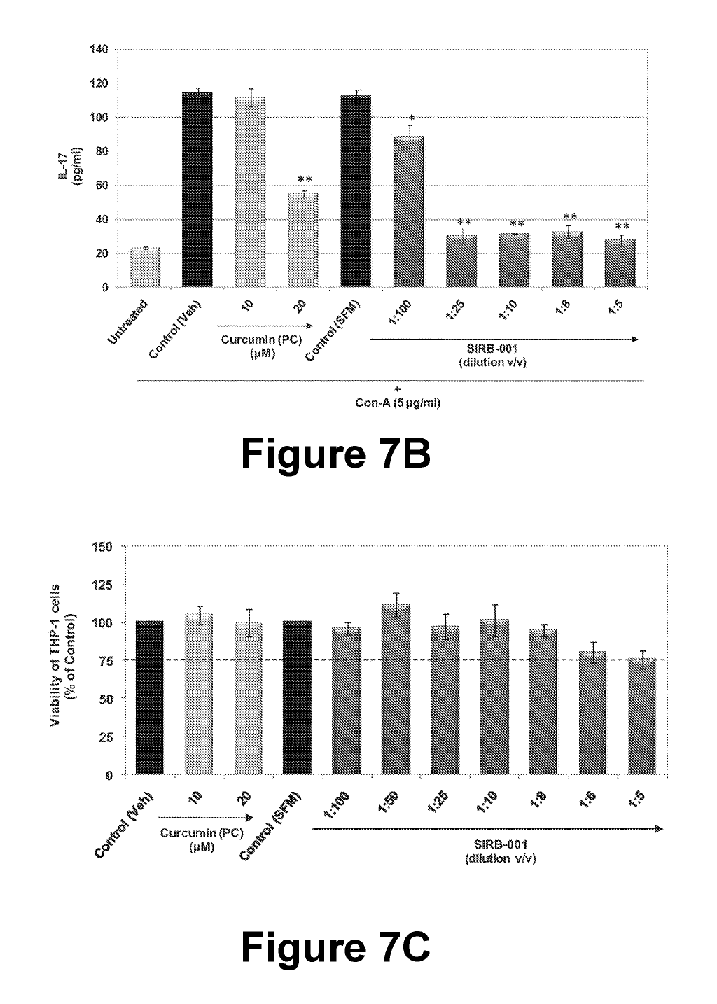

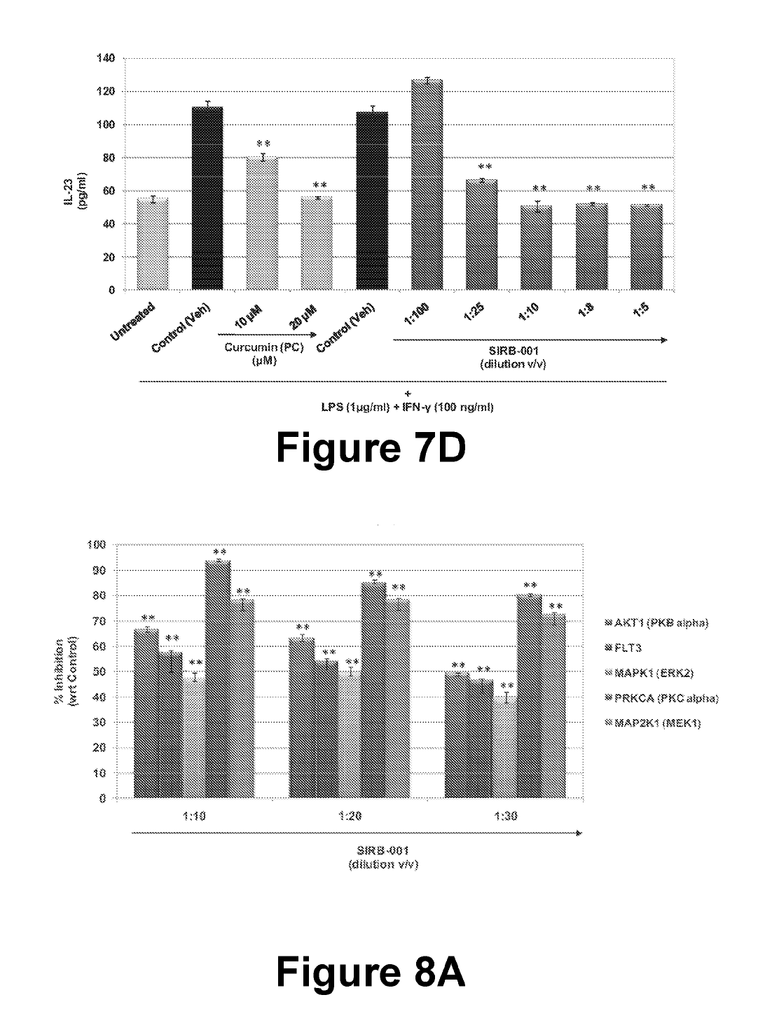

Treatment of murine splenocytes with SIRB-001 (1:100-1:5 v/v) led to >75% cell viability after 24 of treatment (FIG. 7A). For IL-17 estimation, splenocytes were co-incubated with SIRB-001 at dilutions ranging from 1:100-1:5 (v/v) and inflammatory stimulus Con-A (5 μg/ml) for 24 h. Stimulation with Con-A for 24 h resulted in enhanced secretion of IL-17 from basal (untreated) levels of 22.8 pg/ml up to 112.3 pg/ml. SIRB-001 demonstrated significant (p<0.05, p<0.01) alleviation of IL-17 secretion in splenocytes against Con-A stimulated levels alone (FIG. 7B). Curcumin (PC) at 20 μM significantly (p<0.01) inhibited Con-A stimulated IL-17 production.

Inhibitory effect of SIRB-001 on IL-23 secretion was determined in human monocytic cell line (THP-1) against LPS+IFN-stimulated levels. After 24 h of treatment, viability of THP-1 cells was found to be >75% at dilutions ranging from 1:100-1:5 v/v (FIG. 7C). Stimulation of THP-1 cells with LPS+IFN-led to an increase in IL-23 secretion from 55 pg/ml to 107 pg/ml. SIRB-001 was able to exhibit significant (p<0.01) inhibition of IL-23 at dilutions 1:25-1:5 v/v as compared to control cells treated with inflammatory stimulus alone; LPS (1 μg/ml)+IFN-γ (100 ng/ml) (FIG. 7D). Curcumin (PC) at 10 μM and 20 μM significantly (p<0.01) downregulated IL-23 secretion in THP-1 cells.

FIGS. 7A-7D illustrates inhibition of IL-17/IL-23 axis by SIRB-001 in immune cells after 24 h of treatment. (7A) Viability of murine splenocytes (0.2×10⁶ cells/well), (7C) Viability of human monocytes (THP-1 cells, 30,000 cells/well) after 24 h of treatment with SIRB-001. Cell viability was determined by MTT assay and expressed as a percentage (Mean±S.E.M.) of control cells (n=3), (7B) Murine splenocytes (1×10⁶ cells/well) were co-treated with SIRB-001 and Con-A (5 μg/ml), and (7D) THP-1 cells (1×10⁶ cells/well) were co-treated with SIRB-001 and LPS (1 μg/ml)+IFN-γ (100 ng/ml). Secreted levels of IL-17 and IL-23 were estimated by ELISA in culture supernatants. Results are expressed as (Mean±S.E.M. n=3), *: p<0.05, **: p<0.01, significantly different from the Con-A/LPS+IFN-γ treated control group.

### SIRB-001 Targets Signaling Markers in Psoriasis

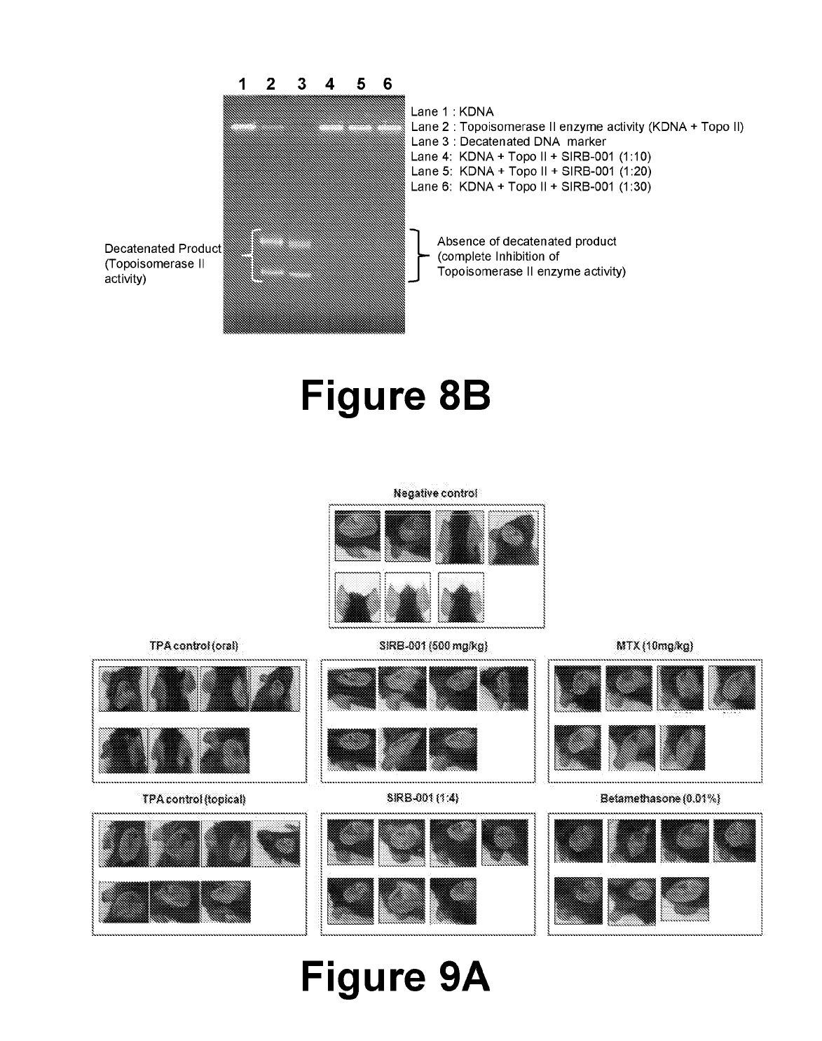

Potential of SIRB-001 to modulate the expression of signaling markers/kinases, which are key members of psoriasis biology was examined at dilutions 1:10, 1:20 and 1:30 v/v (FIG. 8A). SIRB-001 demonstrated significant (p<0.01) inhibitory effect on the expression of AKT1 (PKB alpha), FLT3, MAPK1 (ERK2), PRKCA (PKC alpha) and MAP2K1 (MEK1). SIRB-001 also showed 15% inhibition of NF-_{K}B expression in NFkB-bla THP-1 cells line. In addition, DNA damage/p53 response in p53RE-bla HCT-116 cell line was upregulated by 12% as compared to control. FIG. 8B demonstrates inhibition of Topoisomerase-II activity by SIRB-001. At 1:10, 1:20 and 1:30, SIRB-001 exerted completed inhibition of Topoisomerase-II activity as revealed by absence of decatenated product as compared to control sample (untreated).

FIGS. 8A-8B illustrate A) Inhibitory effect of SIRB-001 on key signaling markers. Results are expressed as (Mean±S.E.M. n=3), **: p<0.01, significantly different from the untreated control group, B) Inhibition of Topoisomerase-II activity by SIRB-001.

### SIRB-001 Displayed Anti-Inflammatory Activity in TPA Induced Ear Inflammation Model

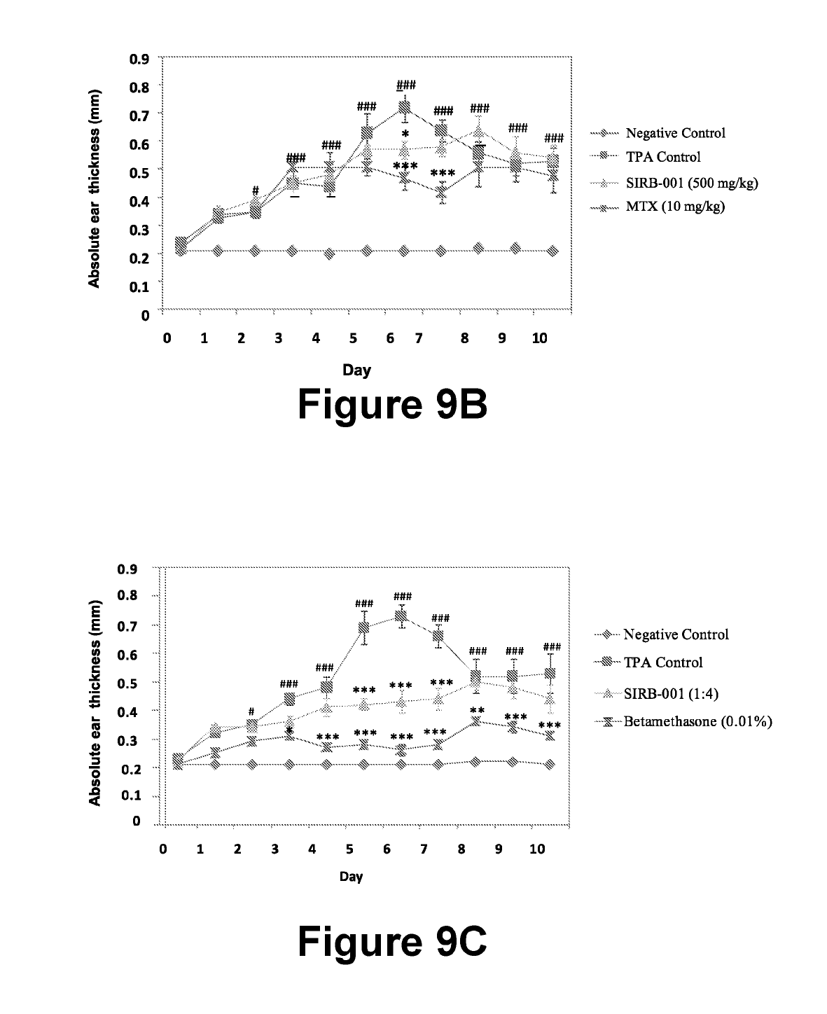

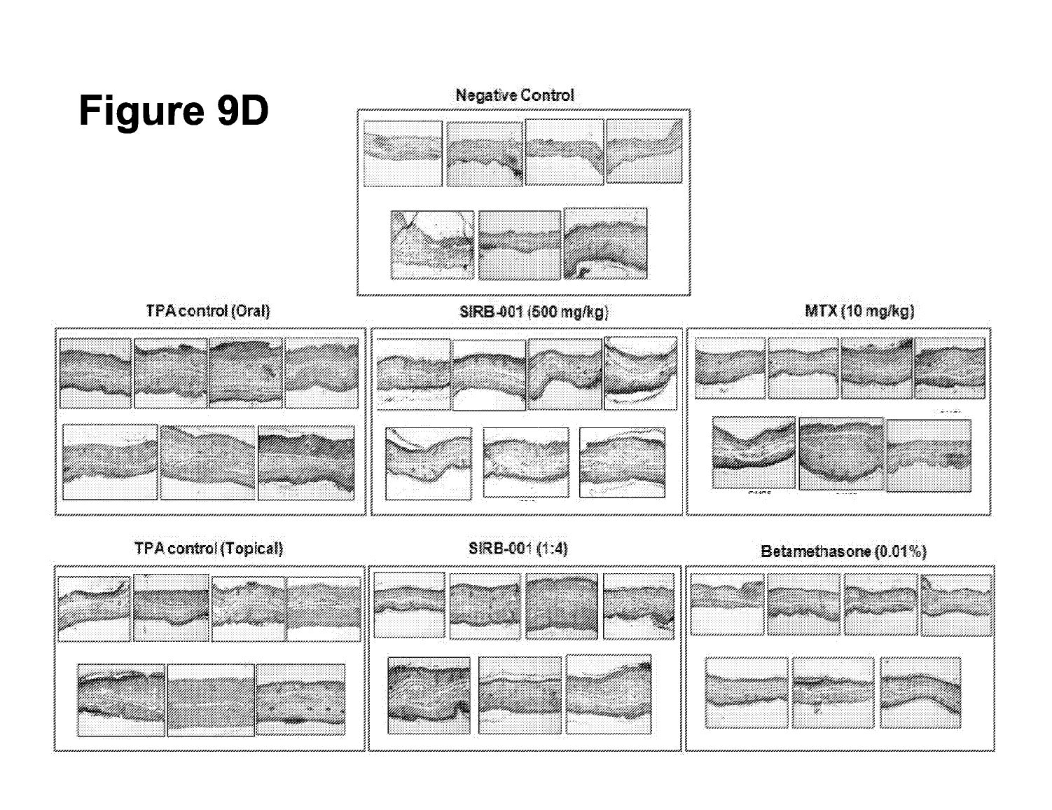

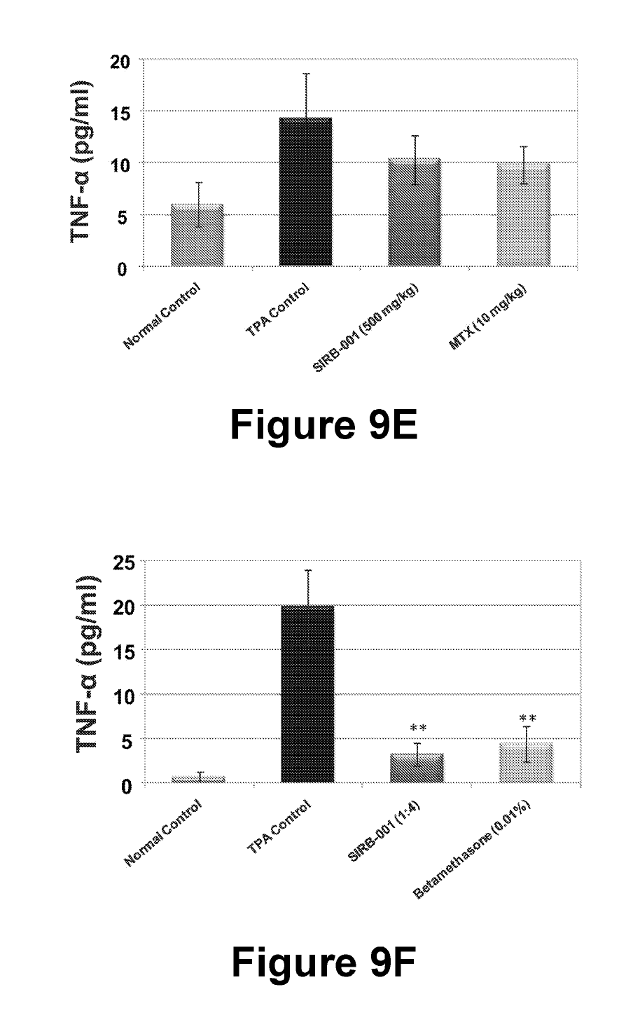

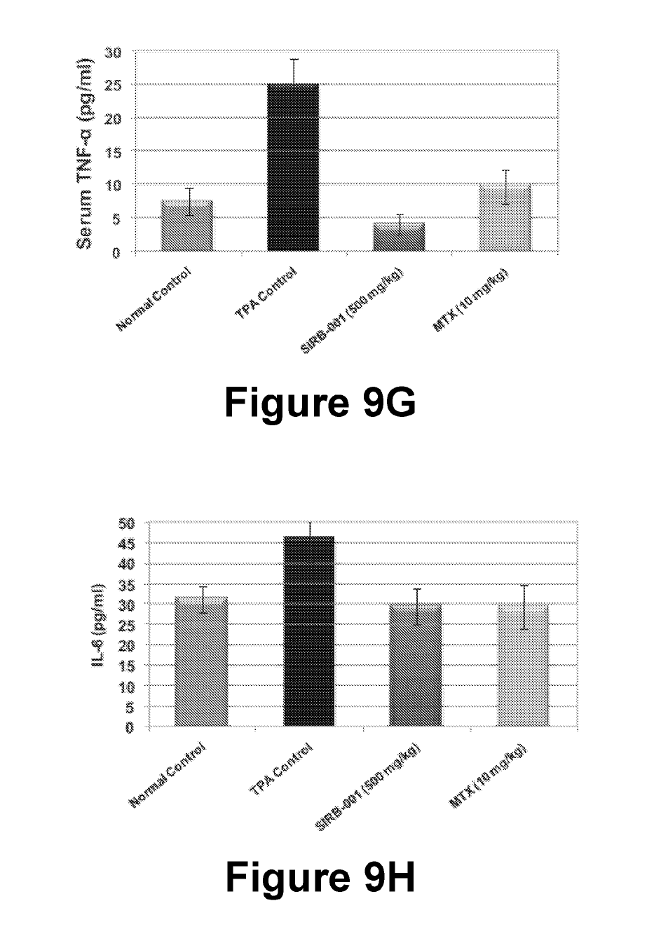

Anti-psoriatic effect of SIRB-001 was evaluated in TPA induced ear inflammation model via reduction of epidermal hyperplasia reflected by changes in ear thickness and inflammatory cytokines in C57BL/6 mice. FIG. 9A represents photographs of the effect of SIRB-001 on TPA induced inflammation.

### Ear Thickness

To evaluate the effect of SIRB-001 on TPA induced inflammation, the absolute ear thickness was measured daily for 11 consecutive days. Significant increase in ear thickness was observed in both the TPA induced disease control groups (oral and topical) on day 2 (p<0.05) and day 3-day 10 (p<0.001), when compared with respective negative control groups. Oral treatment of SIRB-001 at the dose of 500 mg/kg demonstrated significant reduction in absolute ear thickness at day 6 (p<0.05) in comparison with TPA induced disease control group (FIG. 9B). Positive control MTX (10 mg/kg) also resulted in significant reduction of absolute ear thickness at day 6 & 7 (p<0.001). Topical application of SIRB-001 at the concentration of 1:4 significantly decreased TPA induced ear thickness from day 5 to day 7 (p<0.001), when compared with TPA induced disease control group (FIG. 9C). Positive control; 0.01% Betamethasone (PC) also showed significant reduction in absolute ear thickness from day 3 to day 10 (p<0.05; p<0.01; p<0.001).

### Histopathology

FIG. 9D demonstrates the histopathogical changes in SIRB-001 treated animals. In TPA-induced control (oral) group, significant increase (p<0.01) in ear thickness, epidermal ear thickness score and inflammatory cell infiltration score (mild to moderate) was observed. Hyperkeratosis (mild, moderate and severe) and edema (nil to mild) score was also found to be elevated. Oral administration of SIRB-001 (500 mg/mg) resulted in a reduction in ear thickness, hyperkeratosis (nil to severe), edema (mild) and epidermal ear thickness when compared with TPA induced oral disease control. Positive control MTX (10 mg/kg) resulted in significant reduction in epidermal ear thickness score (p<0.01) with marginal effect over ear thickness, hyperkeratosis (mild to moderate) and inflammatory cell infiltration (nil to moderate). Also, in TPA-induced control (topical) group, significant increase (p<0.01) in ear thickness, epidermal ear thickness score and edema score (mild) was observed. Hyperkeratosis (mild, moderate and severe) and inflammatory cell infiltration (mild to moderate) score was also found to be elevated. Topical application of SIRB-001 (1:4) resulted in a marginal reduction in ear thickness, hyperkeratosis (nil to severe score) with significant reduction in epidermal ear thickness score (p<0.01), when compared with TPA induced topical control. Positive control Betamethasone (0.01%) showed significant decrease in ear thickness as well as epidermal ear thickness score (p<0.01) with marginal effect over hyperkeratosis, edema and inflammatory cell infiltration score.

### Secretion of Inflammatory Cytokines (TNF-α, IL-6)

Levels of inflammatory cytokines in ear tissue homogenates of treated animals were estimated by ELISA. Significant increase in TNF-α level was observed in TPA induced oral control (p<0.05) and topical control groups (p<0.01) when compared with negative control group. In animals treated orally with SIRB-001 (500 mg/kg), TNF-α levels were reduced by 27.9% as compared to TPA induced control group (FIG. 9E). MTX (10 mg/kg) used as positive control resulted in inhibition of TNF-α by 31%. Topical application of SIRB-001 (1:4) demonstrated significant (p<0.01) reduction in TNF-α levels by 83.5% as compared to TPA induced control group (FIG. 9F). Positive control; Betamethasone (0.01%) resulted in significant reduction in TNF-α levels in ear tissue homogenate by 77.6% (p<0.01). SIRB-001 (500 mg/kg) also resulted in reduction of serum TNF-α level by 83.4% as compared with TPA induced control (FIG. 9G). MTX (10 mg/kg) inhibited serum TNF-α levels by 60.8%. Topical SIRB-001 (1:4) did not result in any inhibition of serum TNF-α (data not shown).

Levels of IL-6 in ear homogenates were found to be elevated in TPA induced oral and topical control groups as compared to negative control groups. SIRB-001 (500 mg/kg) resulted in inhibition of IL-6 by 35.7% as compared to TPA induced control group (FIG. 9H). MTX (10 mg/kg) as PC inhibited IL-6 levels by 36.2%. Topical application of SIRB-001 (1:4) resulted in downregulation of IL-6 levels by 78.4% as compared to TPA induced control group (FIG. 9I). Positive control; BD (0.01%) resulted in reduction in TNF-α level in ear tissue homogenate by 78%. Serum IL-6 levels were unaffected by SIRB-001 (data not shown).

FIGS. 9A-9I illustrate effects of SIRB-001 on TPA induced ear inflammation in C57BL/6 mice. TPA (100 μg/ml) was applied topically on both right and left ear of mice on day 0, 2, 4, 7 and 9. SIRB-001 was administered to mice orally (500 mg/kg) and topically (1:4) for 10 days. (a) Photographs of mice (day-10), (b-c) Effect of SIRB-001 on absolute ear thickness, (d) Histopathology results. Hyperkeratosis, edema, epidermal ear thickness and inflammatory cell infiltration were scored as nil (−), mild (+), moderate (++) and severe (+++). For scoring of histopathological findings nil, mild, moderate and severe was considered as 0, 1, 2 and 3 respectively. Effect of (e) SIRB-001 (oral; 500 mg/kg) on TNF-α levels in ear homogenates, (f) SIRB-001 (topical; 1:4) on TNF-α levels in ear homogenates, (g) SIRB-001 (oral; 500 mg/kg) on serum TNF-α levels, (h) SIRB-001 (oral; 500 mg/kg) on IL-6 levels in ear homogenates, and (i) SIRB-001 (topical; 1:4) on IL-6 levels in ear homogenates. Results are expressed as (Mean±S.E.M. n=7), *: p<0.05, **: p<0.01, ***: p<0.001 significantly different from the TPA-induced control group, #: p<0.05, ###: p<0.001 significantly different from the normal control group.

### SIRB-001 Showed Anti-Psoriatic Activity in IMQ Induced Model

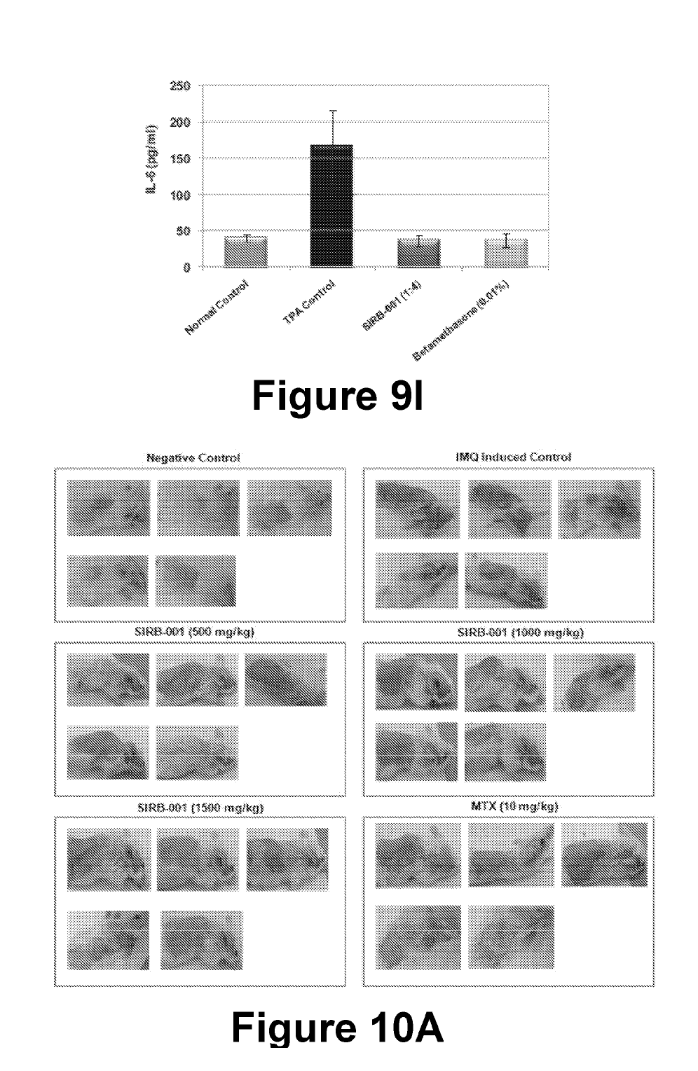

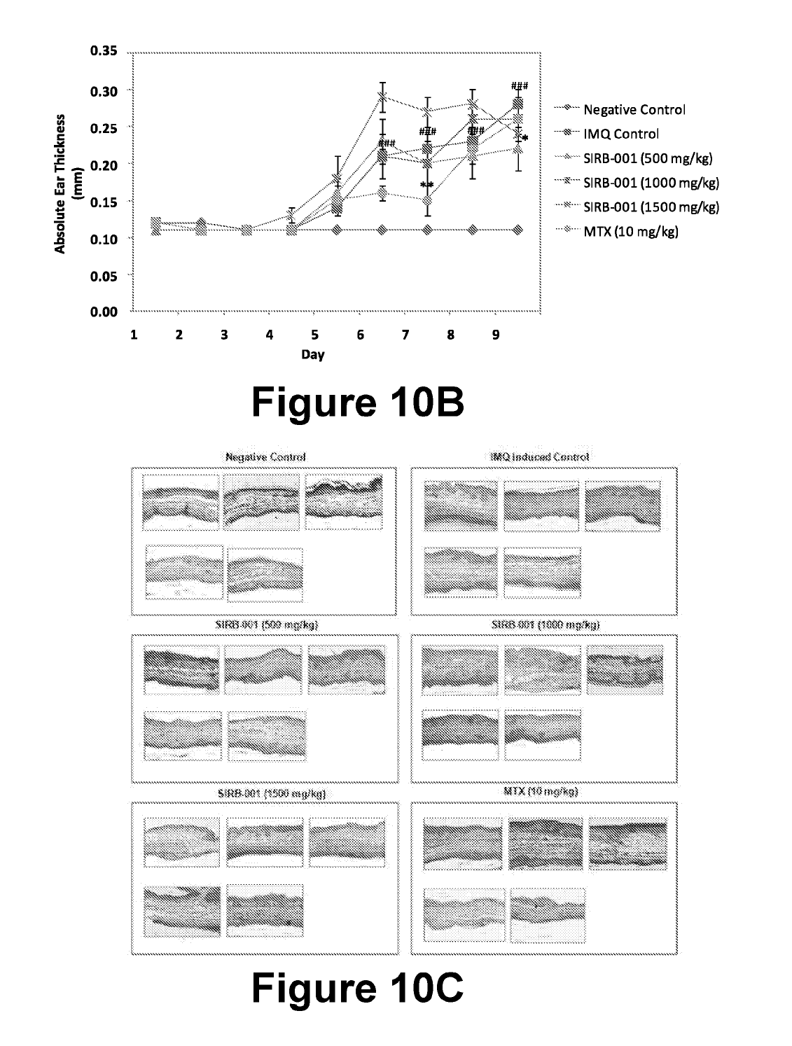

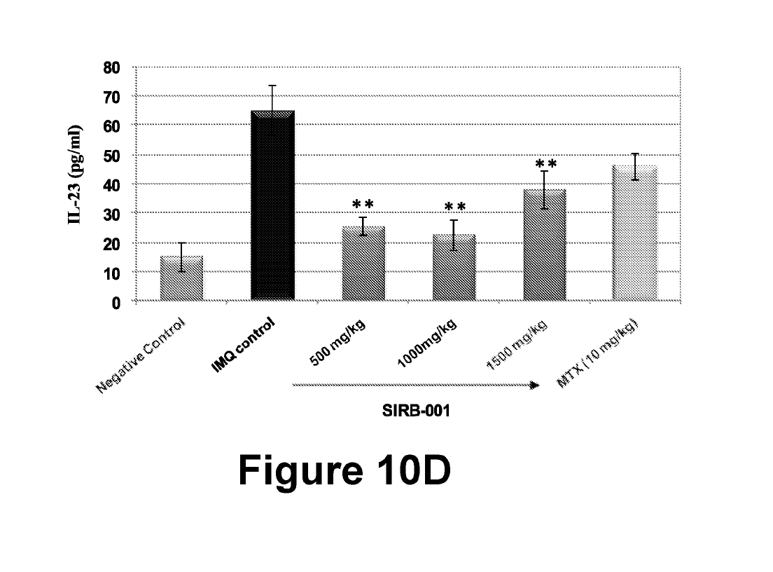

Anti-psoriatic effect of SIRB-001 was evaluated in IMQ induced psoriasis model in Balb/c mice. Photographs representing the effect of SIRB-001 on IMQ induced inflammation in mice ear and dorsal areas are shown in FIG. 10A. Repeated application of IMQ in right ear and dorsal skin of mice demonstrated profound erythrema, redness, scaling and thickening.

### Ear Thickness

The absolute ear thickness was determined daily for 9 days. Significant increase (p<0.001) in ear thickness was observed in the IMQ induced control group from day 6 to day 9, when compared with normal control. SIRB-001 (500 mg/kg) resulted in significant reduction of absolute ear thickness on Day 9 (p<0.01) when compared with IMQ induced control. Among the three tested oral doses of SIRB-001, low dose (500 mg/kg) showed maximum effect. MTX 10 mg/kg (used as positive control) resulted in significant reduction of absolute ear thickness on Day 7 (p<0.01) (FIG. 10B).

### Histopathology

The H&E-stained ear sections of all the experimental groups are represented in FIG. 10C. Oral disease control showed significant increase in epidermal ear thickness score (p<0.01), edema (p<0.05) (mild to moderate) and inflammatory cell infiltration (p<0.01) (mild to severe). Ear thickness was also found to be increased however no change in hyperkeratosis (mild to moderate) was found when compared with oral normal control. Maximum effect over histopathological scores was exerted by low dose SIRB-001 (500 mg/kg). Treatment with low dose (500 mg/kg) of SIRB-001 showed mild hyperkeratosis, mild to moderate edema and mild to severe inflammatory cell infiltration whereas no effect was found over epidermal ear thickness score. Treatment with SIRB-001 (1000 mg/kg) showed mild to moderate hyperkeratosis and edema but no effect over inflammatory cell infiltration and epidermal ear thickness score. SIRB-001 (1500 mg/kg) showed no effect over hyperkeratosis (mild to moderate), edema (mild to moderate), inflammatory cell infiltration (mild to severe), epidermal ear thickness score and ear thickness. MTX (10 mg/kg) exerted better effect over edema (nil to mild score), partial effect over epidermal ear thickness, hyperkeratosis (mild to moderate score) and inflammatory cell infiltration (nil to moderate score).

### Secretion of IL-17, IL-23

IL-17 and IL-23 levels were analyzed in ear tissue homogenates by ELISA. With IMQ induction, levels of IL-23 in ear tissue homogenates were found to be increased significantly (p<0.01) as compared to normal control group. SIRB-001 at doses of 500 mg/kg, 1000 mg/kg and 1500 mg/kg resulted in inhibition of IL-23 by 60.8%, 65.2% and 41.7% (p<0.01) as compared to IMQ-treated Control group (FIG. 10D). MTX (10 mg/kg) resulted in inhibition of IL-23 by 29.4%. Levels of IL-17 were found to be remarkably up-regulated in IMQ-induced control group in comparison with normal control. But no effect on IL-17 secretion was observed in any of the SIRB-001 treatment groups (data not shown).

FIGS. 10A-10D. Effect of SIRB-001 on IMQ induced psoriasis in Balb/c mice. IMQ (5%) was applied topically each on dorsal area (shaved back) and right ear of mice for 8 days. SIRB-001 was administered orally at dose of 500 mg/kg, 1000 mg/kg and 1500 mg/kg daily. (a) Photographs of mice (day-9), (b) Effect of SIRB-001 on absolute ear thickness, (c) Histopathology results. Hyperkeratosis, edema, epidermal ear thickness and inflammatory cell infiltration are scored as nil (−), mild (+), moderate (++) and severe (+++), and (d) IL-23 levels in ear tissue homogenates. Results are expressed as (Mean±S.E.M. n=5), *: p<0.05, **: p<0.01, ***: p<0.001 significantly different from the TPA-induced control group, ###: p<0.001 significantly different from the normal control group.

Despite of the extensive research being conducted across the world, psoriasis still remains to be a chronic, autoimmune, inflammatory, hyperproliferative and scaling skin disorder with a complex pathophysiology and less understood etiology. In its advanced stages, psoriasis can be a disabling disease and has long been associated with severe psychological, social and economical consequences. In some instances, this skin disorder may become even life-threatening to patients (Gudjonsson and Elder, 2007). Presently, large number of conventional anti-psoriatic treatments is available in market including topical agents (Vitamin D, calcipotriol, corticosteroids, dithranol and retinoids), systemic drugs (methotrexate and cyclosporine etc.), phototherapy (UV-B, Psoralen plus ultraviolet therapy and excimer laser etc.), combination therapy, herbal therapy and other novel molecules (Rahman et al., 2012). However, multiple adverse side effects are known to be associated with these therapies, such as organ toxicity, carcinogenicity, immunosuppression along-with inadequate efficacy and inconvenient administration schedules. These limitations of conventional treatments have led to discovery of a new series of biologic agents (TNF-α inhibitors, Etanercept, infliximab, Efalizumab etc.) which target the pathological process of psoriasis with very high specificity (Cather and Crowley, 2014; Rich and Bello-Quintero 2004). Therefore, identification and development of new anti-psoriatic drugs with high efficacy and enhanced safety has emerged as a hot area of research presently. The increasing demand for the search of new, efficacious and alternative therapeutic agents for psoriasis with minimum side-effects has fueled the development of herbal based medicines as a stand-alone or combinatorial agents (Deng et al., 2013; May et al., 2012).

Interestingly, Traditional Chinese Medicine has gained special attention of researchers on account of demonstrating efficacious results in the management and treatment of psoriasis (Li et al., 2012). The pharmacological action of TCM based medicines has been investigated in combination with standard mode of therapies (Baroni et al., 2014; Yang et al., 2015). TCM has secured a special place as anti-inflammatory therapeutic agent for various skin orders, which are associated with inflammation (Chan et al., 2008). The emerging beneficial role of TCM in inflammatory skin disorders had prompted and encouraged us to investigate the extensive mechanistic mode of action of SIRB-001 in one of the most crucial inflammatory skin-disorders; psoriasis. SIRB-001 is a novel herbal concoction developed by us, which comprises of 3 traditional Chinese herbs in ratio of 1:1:3; Da Huang (Radix et Rhizoma Rhei), Sheng Di Huang (Radix Rehmanniae) and Jin Yin Hua (Flos Lonicerae). Although beneficial effects of these constituent herbs in various inflammatory disorders including psoriasis have been vastly reported (Moon et al., 2006, Viljoen et al., 2012; Kang et al., 2014), their detailed and specific, cellular mechanistic modes of action are yet to be fully elucidated for anti-psoriatic potential. Targeted effects of SIRB-001 on crucial arms of psoriasis, such as hyper-proliferation of keratinocytes, apoptosis, inflammation, and angiogenesis were studied employing appropriate cell based assays and animal models.

Psoriasis is a disturbing, chronic and inflammatory skin disorder, which is classically associated with uncontrolled hyperproliferation and abnormal differentiation of keratinocytes, epidermal hyperplasia and enhanced migration and infiltration of inflammatory cells in the areas of epidermis and dermis (Boehncke, 2015; Pol et al., 2003). To assess the anti-psoriatic potential of SIRB-001 in keratinocytes via most critical arms viz., inhibition of proliferation, induction of apoptosis and inhibition of inflammatory molecules and angiogenic molecules, we employed HaCaT, which is a spontaneously transformed and immortalized normal human keratinocyte cell line and widely used as a suitable cell model system to study epidermal hyper-proliferation in psoriasis studies (Hollywood et al., 2015). In addition, immune cells such as murine splenocytes and THP-1 (human monocytes) were used to study IL-17/IL-23 secretion.

Hyperproliferation along-with an imbalanced apoptotic mechanism in keratinocytes constitute the key factor driving the onset of psoriasis. Hence, a number of compounds possessing anti-proliferative activity on the growth of keratinocytes in conjunction with pro-apoptotic potential present as promising candidates for development as anti-psoriatic agents for effective management of psoriasis. In the present study, SIRB-001 demonstrated significant and dose dependent anti-proliferative effect in HaCaT cells after 48 h of treatment. Growth-inhibitory potential of SIRB-001 was further substantiated with noticeable alterations induced in cellular morphology of HaCaT cells. Characteristic features of apoptotic cells with enhanced cell-detachment and ballooning were observed with increasing concentrations of SIRB-001, which were prominently distinct from untreated, control cells. Inhibition of cellular proliferation and remarkable changes in cellular morphology strongly indicated anti-psoriatic potential of SIRB-001. To delineate the pro-apoptotic properties of SIRB-001 in HaCaT cells, effects on a panel of early, mid and late apoptotic markers were evaluated. SIRB-001 demonstrated significant apoptotic potential by inducing an increase in the activity of all the apoptotic markers tested. This effect was mediated by a substantial increase in early apoptotic markers such as Annexin-V staining and loss of mitochondrial potential by JC-1 staining, stimulation of mid phase markers such as Caspase-3 activity and significant activation of late markers such as sub-(G0/G1) population in cell cycle, and enhanced DNA fragmentation. These results suggest that the anti-proliferative effect of SIRB-001 may be attributed to the induction of apoptosis, which reflects its anti-psoriatic potential by effectively controlling the abnormal growth and proliferation of keratinocytes. An inhibition of keratinocytes proliferation with pro-apoptotic potential strongly indicates the anti-psoriatic activity of SIRB-001. Likewise, many anti-psoriatic herbal and synthetic compounds are reported to demonstrate anti-proliferative and pro-apoptotic activity in keratinocytes. Artemether, semi-synthetic derivative of artemisinin isolated from A. annua exhibits anti-psoriatic activity at cellular levels via inhibition of HaCaT cell proliferation and induction of apoptosis (Wu et al., 2015). Novel antipsoriasis codrug, Naproxyl Dithranol also exerts inhibition of keratinocytes proliferation and enhances apoptosis (Lau et al., 2011). Similarly, topical corticosteroids have been widely shown to activate various markers of apoptosis and display proliferation inhibition of HaCaT cells (Guichard et al., 2015). Herbal agents have been extensively reported to exhibit inhibition of keratinocytes proliferation with induction of apoptosis, such as indigo naturalis (Lin et al., 2009) and Rubia cordifolia L extract (Lin et al., 2010). A library of 60 chinese medicinal herbs was assessed for efficacy in psoriasis by inhibitory effect on HaCaT cells proliferation (Tse et al., 2006).

Excessive production of inflammatory markers by keratinocytes and infiltrating immune cells is another pivotal feature contributing to the progress and development of psoriasis. Cytokines secreted by keratinocytes are key players responsible for the migration of increased number of immune cells towards the site of inflammation leading to hyperproliferation of keratinocytes (Marble et al., 2007). TNF-α plays a major role in the pathogenesis of psoriasis by acting as the most detrimental factor in the network of inflammatory cytokines. TNF-α recruits and stimulates the activity of neutrophils, which are a major component of the inflammatory infiltrating cells in psoriasis (Yost and Gudjonsson, 2009). TNF-α also results in activation of cascades leading to increased secretion of other pro-inflammatory cytokines such as IL-8, ICAM-1, VCAM-1 etc. (Terajima et al., 1998). It has been widely used to induce inflammatory conditions in keratinocytes in vitro to mimic the psoriatic inflammation (Brotas et al., 2012). IFN-γ produced by activated Th1 cells and dermal dendritic cells is another principle cytokine of cell-mediated immunity, which plays prominent role in psoriasis by stimulating production of other key inflammatory molecules (Kryczek et al., 2008). Thus, the cytokines and inflammatory markers such as TNF-α, IFN-γ, IL-6, NO and sPLA₂ present to be interesting therapeutic interventions for anti-psoriatic agents. In our study also, SIRB-001 exerted a strong inhibitory effect on the secretion of key pro-inflammatory cytokines (TNF-α and IFN-γ) in HaCaT cells activated with inflammatory stimulus TNF-α. A moderate inhibition of IL-6 was induced by SIRB-001 against TNF-α stimulated levels. Other inflammatory markers playing important role in psoriasis such as NO and sPLA₂ were also remarkably downregulated in HaCaT cells. On account of the prominent intervention of the network of pro-inflammatory mediators involved in the pathogenesis of psoriasis, it is strongly indicated that SIRB-001 possesses promising anti-psoriatic potential mediated via blockade of the cytokines arm in addition to inhibition of proliferation and induction of apoptosis in keratinocytes. This effect is correlated with various anti-psoriatic agents reported in literature, which are known to display anti-inflammatory activity via inhibition of cytokines and other markers. Thai medicinal herb extracts have demonstrated an inhibition of NF-κB signaling in keratinocytes (Saelee et al., 2011). Traditional herbal formula such as Jakyakgamcho-tang (Paeonia lactiflora and Glycyrrhiza uralensis) demonstrated anti-psoriatic potential supported with in vitro downregulation of chemokines, NF-κB and STAT1 inhibition in TNF-α+IFN-γ stimulated HaCaT cells (Jeong et al., 2015). In addition, luteolin is reported to inhibit IL-6, IL-8 and VEGF secretion from TNF-α triggered HaCaT keratinocytes (Weng et al., 2014).

Angiogenesis is another important arm in psoriasis, which is recognized for aiding the progress of this disease (Li et al., 2014). Based on the relevance of angiogenesis in psoriasis, we investigated the anti-angiogenic potential of SIRB-001 by estimating VEGF secretion in HaCaT cells. Interestingly, a significant downregulation of VEGF production against the basal (untreated) levels was observed in HaCaT cells. This observation extends the portfolio of SIRB-001 as an anti-psoriatic agent, strongly supplementing its anti-proliferative, pro-apoptotic and anti-inflammatory action. These encouraging results are in line with recent research carried out by various groups, such as Gambogic acid has been shown to exhibit anti-psoriatic potential through inhibition of angiogenesis and inflammation (Wen et al., 2014). Shikonin exerts its anti-psoriatic activity via suppression of IL-17 induced VEGF expression in HaCaT cells (Xu et al., 2014). These findings suggest the importance of VEGF downregulation in the angiogenic arm for therapeutic efficacy of anti-psoriatic drugs.

In addition, IL-17/IL-23 axis in the immune arm has been lately reported as a key contributor towards the development of various inflammatory disorders including psoriasis (Toussirot, et al., 2012; Lowes et al., 2013). Recently, IL-17/IL-23 axis has been targeted by new generation of anti-psoriatic agents including biologics such as Ustekinumab, Secukinumab, Ixekizumab, Brodalumab, Guselkumab and Tildrakizumab (Mease, 2015). Realizing the emerging role of IL-17/IL-23 axis in psoriasis, the specific inhibitory action of SIRB-001 on this arm was also explored using immune cell populations. SIRB-001 resulted in significant inhibition of Con-A stimulated levels of IL-17 secretion in murine splenocytes. Also, significant downregulation of LPS+IFN-γ induced secretion of IL-23 was shown by SIRB-001 in THP-1 cells (human monocytes). Strong inhibitory potential of SIRB-001 on IL-17/IL-23 axis suggests its effective role in curbing the inflammatory switch, which regulates the onset and progress of psoriasis.

Complex signaling cascades and pathways govern the pathogenesis of psoriasis, which are still being investigated in great depths by researchers (O'Rielly and Rahman, 2011). PI3K-AKT-mTOR kinase system has recently emerged as the underlying signaling pathway in psoriasis (Raychaudhuri and Raychaudhuri, 2014). In this study, signaling kinases and other molecules contributing towards the pathogenesis and mechanism of psoriasis were tested for any possible modulation under the influence of SIRB-001. Remarkable inhibition of signaling kinases such as AKT1 (PKB alpha), FLT3, MAPK1 (ERK2), PRKCA (PKC alpha) and MAP2K1 (MEK1) was observed with SIRB-001. In addition, SIRB-001 also possessed inhibitory potential on NF-κB expression and activation of DNA damage/p53 response. Complete inhibition of Topoisomerase-II activity was shown by SIRB-001. These interesting results via inhibition of multiple signaling markers of psoriasis by SIRB-001 highlight its role in the modulation of key players of psoriasis and its possible efficacy.

The anti-psoriatic efficacy observed in vitro was also evaluated in animal based models in vivo. SIRB-001 when tested orally and topically, also demonstrated anti-inflammatory effects in TPA induced model of psoriasis in C57BL/6 mice. SIRB-001 was able to remarkably inhibit TPA induced ear thickness, TNF-α levels in ear and serum and IL-6 levels in ear homogenates. These results were consistent with histopathogical findings. SIRB-001 tested orally at 500 mg/kg also demonstrated efficacious effects in IMQ induced model in Balb/c mice. Inhibition of ear thickness and downregulation of IL-23 levels by SIRB-001 were observed, which were in line with histopathological results. These results are in accordance with the reported data for herbal medicines. Chinese herbal medicine (Tuhuai extract) with anti-psoriatic potential has been shown to demonstrate anti-inflammatory activity in TPA-induced murine model (Man et al., 2008). Recently, Glycyrrhizin has shown efficacious effects in Imiquimod-induced psoriasis-like skin lesions in BALB/c Mice (Xiong et al., 2015). A chinese Formula Jueyin Granules have exhibited anti-psoriatic potential in IMQ induced model in mice (Ma et al., 2014).

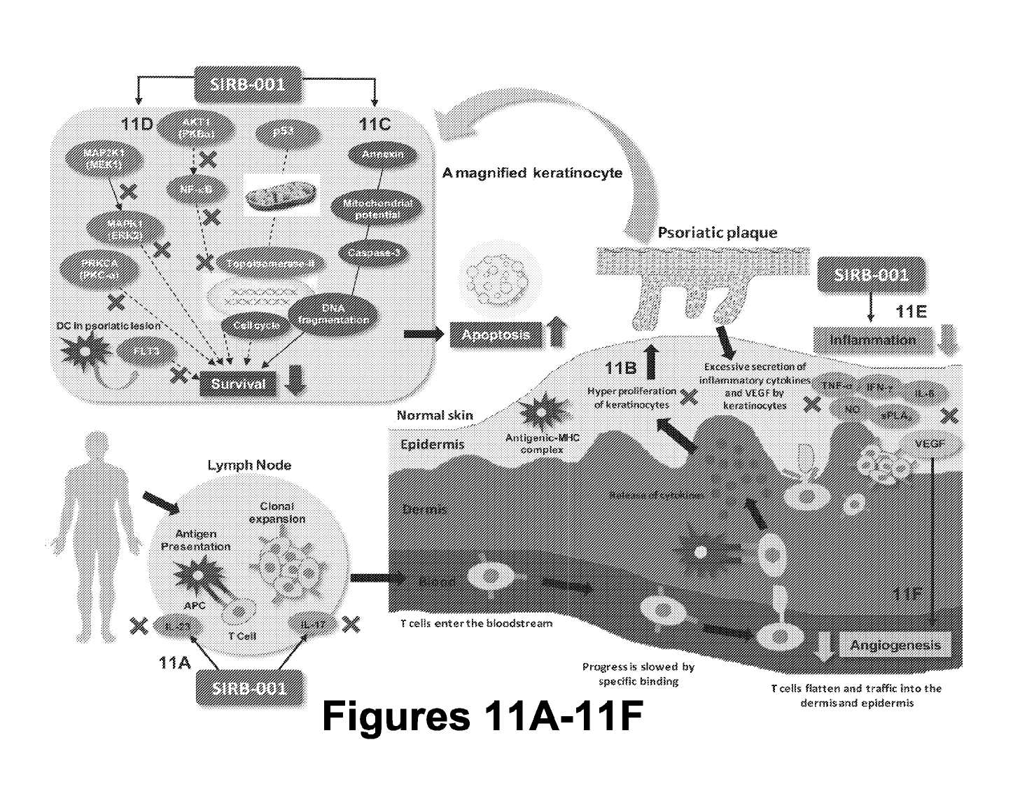

In conclusion, SIRB-001 has exhibited potential anti-psoriatic properties in vitro in the cell based models utilizing keratinocytes and immune cells, and in cell-free enzyme based models. SIRB-001 has also demonstrated in vivo efficacy in mice models. The multifaceted anti-psoriatic action of SIRB-001 is revealed by targeting all the hallmark features of psoriasis, such as excessive proliferation, inflammation, angiogenesis in keratinocyte arm, IL-17/IL-23 axis in immune arm and key signaling markers playing crucial role in psoriasis. Based on the interesting and encouraging results obtained in the study, FIGS. 11A-11F summarizes the detailed mechanistic action of SIRB-001 on specific arms and markers playing important role in the onset, progression and development of psoriasis. Considering the diverse array of anti-psoriatic properties demonstrated, SIRB-001 presents as a promising TCM based therapeutic agent for psoriasis and highlights the further scope of development as an anti-psoriatic drug.

FIGS. 11A-11F Schematic representation of mechanistic action of SIRB-001 in onset, progress and development of psoriasis. (11A) SIRB-001 targets the immune arm via inhibition of IL-17/IL-23 axis, (11B) SIRB-001 exerts inhibition of abnormal keratinocytes proliferation and, (11C) Pro-apoptotic potential of SIRB-001 via early, mid and late markers; Annexin-V, Mitopotential, Caspase-3, Cell cycle and DNA fragmentation, (11D) SIRB-001 exhibits modulation of key signaling molecules [AKT1 (PKB alpha), FLT3, MAPK1 (ERK2), PRKCA (PKC alpha), MAP2K1 (MEK1), p53 and NF-_{K}B] and Topoisomerase-II, (11E) Inhibitory effect of SIRB-001 on key pro-inflammatory cytokines such as TNF-α, IFN-γ, IL-6 and other inflammatory molecules such as NO and sPLA₂ in keratinocytes shows the anti-inflammatory role of SIRB-001, (11F) Secretion of VEGF by keratinocytes is also inhibited by SIRB-001, highlighting its anti-angiogenic potential. (Figure adapted from Papp, 2004).

### MULTIPLEX EXAMPLES

TH1, TH17 and TH22 cytokines play key roles in the pathogenesis of psoriasis. Combinations of markers may be used in a diagnostic test kit or in a prognostic test kit or both. These markers may include any of the following 15-plex (TH17):

| | |
|---|---|
| 1 | IL-1β |
| 2 | IL-4 |
| 3 | IL-6 |
| 4 | IL-10 |
| 5 | IL-17A |
| 6 | IL-17F |
| 7 | IL-21 |
| 8 | IL-22 |
| 9 | IL-23 |
| 10 | IL-25 |
| 11 | IL-31 |
| 12 | IL-33 |
| 13 | IFN-γ |
| 14 | sCD40L |
| 15 | TNF-α |

Table 1 is an example combination of 15 markers that may be used in a diagnostic test kit or in a prognostic test kit or both.
The markers may include any of the following 27-plex:

| | |
|---|---|
| 1 | FGF basic |
| 2 | Eotaxin |
| 3 | G-CSF |
| 4 | GM-CSF |
| 5 | IFN-γ |
| 6 | IL-1β |
| 7 | IL-1ra |
| 8 | IL-2 |
| 9 | IL-4 |
| 10 | IL-5 |
| 11 | IL-6 |
| 12 | IL-7 |
| 13 | IL-8 |
| 14 | IL-9 |
| 15 | IL-10 |
| 16 | IL-12 (p70) |
| 17 | IL-13 |
| 18 | IL-15 |
| 19 | IL-17 |
| 20 | IP-10 |
| 21 | MCP-1 (MCAF) |
| 22 | MIP-1α |
| 23 | MIP-1β |
| 24 | PDGF-BB |
| 25 | RANTES |
| 26 | TNF-α |
| 27 | VEGF |

Table 2 is an example combination of 27 markers that may be used in a 27-plex or smaller subset within a diagnostic or prognostic kit.
The markers may include any of the following 35 plex:

| S. No. | Biomarker |
|---|---|
| 1 | IL-1β |
| 2 | IL-4 |
| 3 | IL-6 |
| 4 | IL-10 |
| 5 | IL-17A |
| 6 | IL-17F |
| 7 | IL-21 |
| 8 | IL-22 |
| 9 | IL-23 |
| 10 | IL-25 |
| 11 | IL-31 |
| 12 | IL-33 |
| 13 | IFN-γ |
| 14 | sCD40L |
| 15 | TNF-α |
| 16 | FGF basic |
| 17 | Eotaxin |
| 18 | G-CSF |
| 19 | GM-CSF |
| 20 | IL-1ra |
| 21 | IL-2 |
| 22 | IL-5 |
| 23 | IL-7 |
| 24 | IL-8 |
| 25 | IL-9 |
| 26 | IL-12 (p70) |
| 27 | IL-13 |
| 28 | IL-15 |
| 29 | IP-10 |
| 30 | MCP-1(MCAF) |
| 31 | MIP-la |
| 32 | MIP-1β |
| 33 | PDGF-BB |
| 34 | RANTES |
| 35 | VEGF |

Table 3 is an example combination of 35 markers that may be used in 35 plex. These 35 markers or subsets of these markers may be used in a diagnostic or prognostic test kit.

The Bio-Plex® assays are built around the well known Luminex xMAP technology using a bead-based flow cytometric platform dedicated to multiplex analysis. A Bio-Plex Human Cytokine, Chemokine, and Growth Factor Magnetic Bead-Based Assays detect and measure levels of multiple analytes in diverse matrices such as serum, plasma, and tissue culture supernatants.

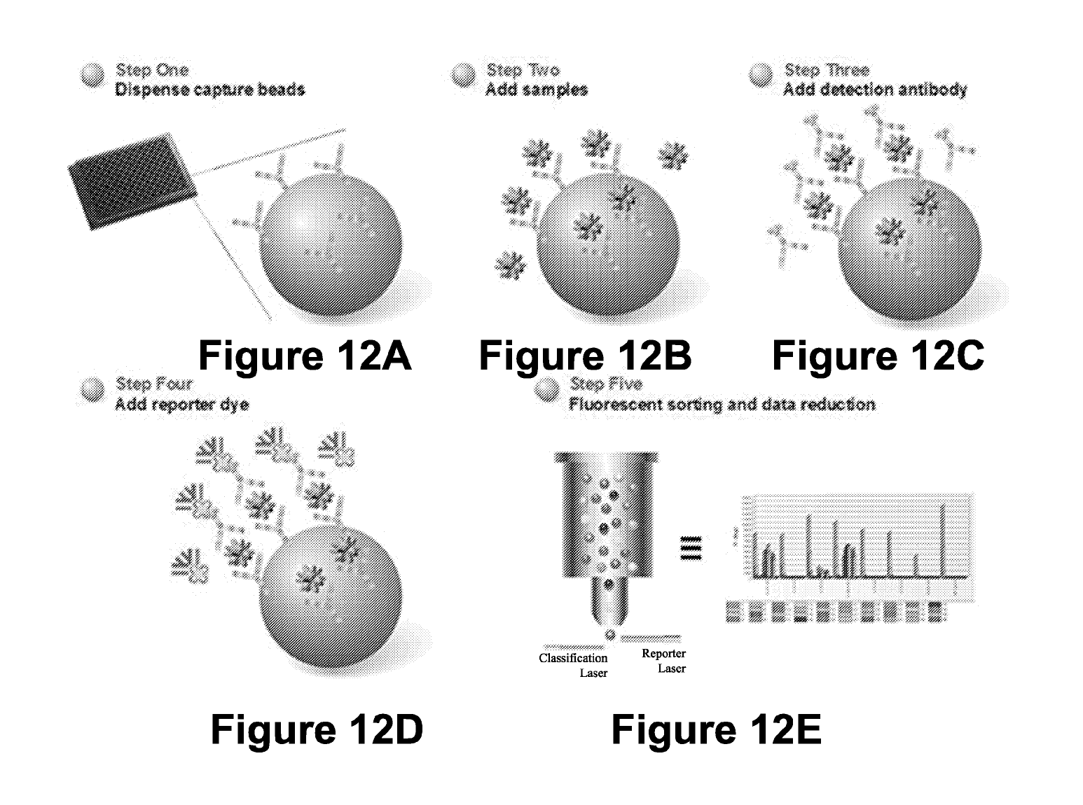

Similar to ELISA, a majority of assays are designed according to a capture sandwich immunoassay format. Briefly, the capture antibody-coupled beads are first incubated with antigen standards or samples for a specific time. The plate is then washed to remove unbound materials, followed by incubation with biotinylated detection antibodies. After washing away the unbound biotinylated antibodies, the beads are incubated with a reporter streptavidin-phycoerythrin conjugate (SA-PE). Following removal of excess SA-PE, the beads are passed through the array reader, which measures the fluorescence of the bound SA-PE (FIG. 12).

FIGS. 12A-12E schematically illustrates an immunoassay sandwich-based assay workflow.

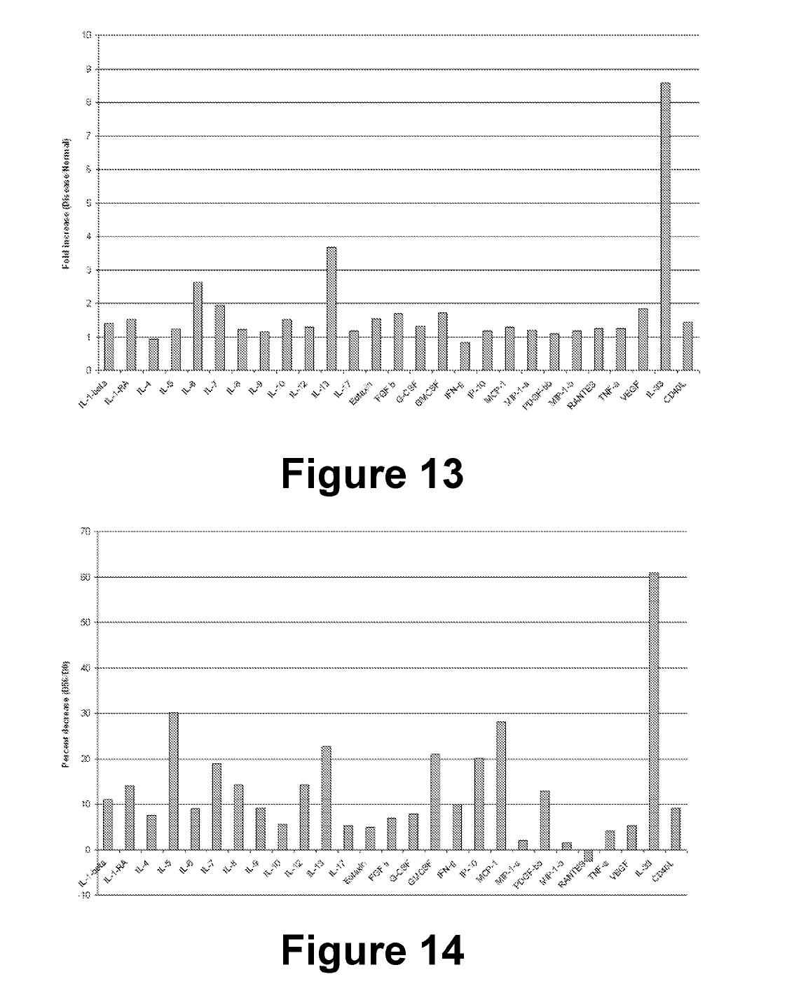

Serum samples of 30 patients obtained at 2 time points 0 and 56 days and 7 normals were analyzed for 35 markers using 2 Bioplex assay kits. FIGS. 13-14 illustrate effects of psoriasis on certain markers compared to healthy controls. At least a 1.5 fold increase in levels were observed in 7 markers in disease condition as compared to normal. Those 7 markers are as follows:

| | |
|---|---|
| 1 | IL-6 |
| 2 | IL-7 |
| 3 | IL-13 |
| 4 | FGF-B |
| 5 | GMCSF |
| 6 | VEGF |
| 7 | IL-33 |

Table 4 is an example combination of 7 markers that may be measured in a diagnostic or prognostic test kit. Subsets of these 7 markers and other markers may also be used.

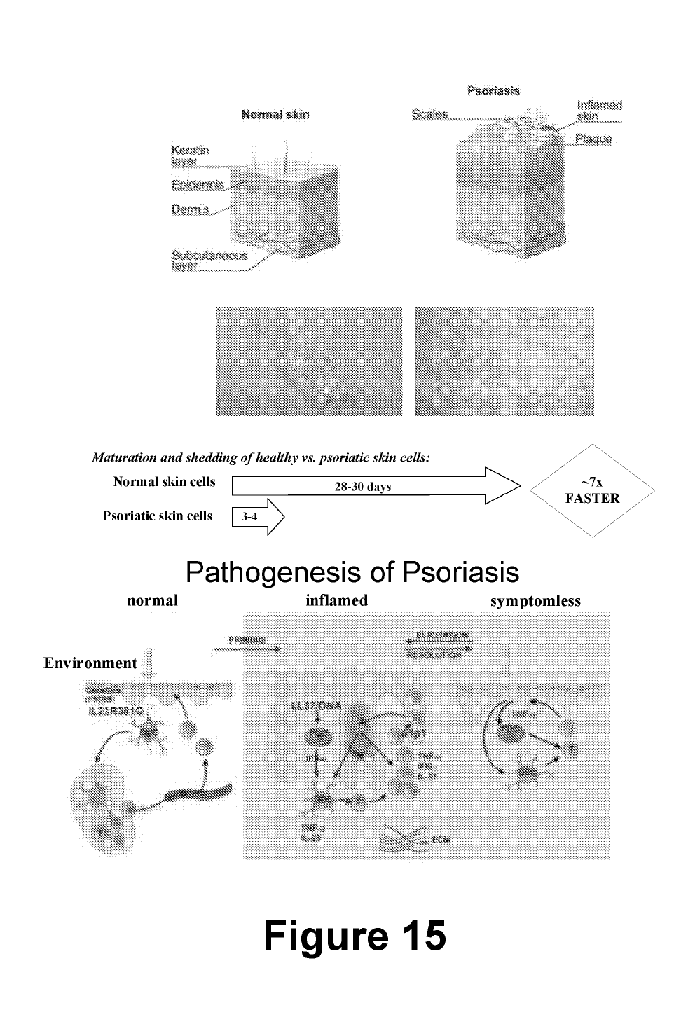

FIG. 15 illustrates a pathogenesis of psoriasis. Psoriasis is a chronic inflammatory, multi-system disease associated with considerable morbidity and co-morbid conditions. Psoriasis affects approximately 2% of the world's population. The severity of psoriasis is defined by extent of body surface area involvement, involvement of localized body regions such as flexures, hands, feet, facial, and genital regions which, may interfere significantly with activities of daily life and have a substantial psychological impact on one's personal well-being. In psoriasis, the immune system is mistakenly activated, which leads to overproduction of skin cells. Skin cells build up too rapidly on the surface of the skin, forming raised, red, scaly patches (called plaques). These plaques are often itchy and sometimes painful. The causes of psoriasis are not fully understood today. The current consensus is that immune system, genetics and the environment (e.g., stress, cold weather) all play major roles in the development of psoriasis. As a result there is skin inflammation accompanied by overproduction of skin cells. Cells in the upper skin layer normally mature and are shed from the skin's surface every 28 to 30 days. With psoriasis, the cells can mature in 3 to 6 days then move to the skin and pile up.

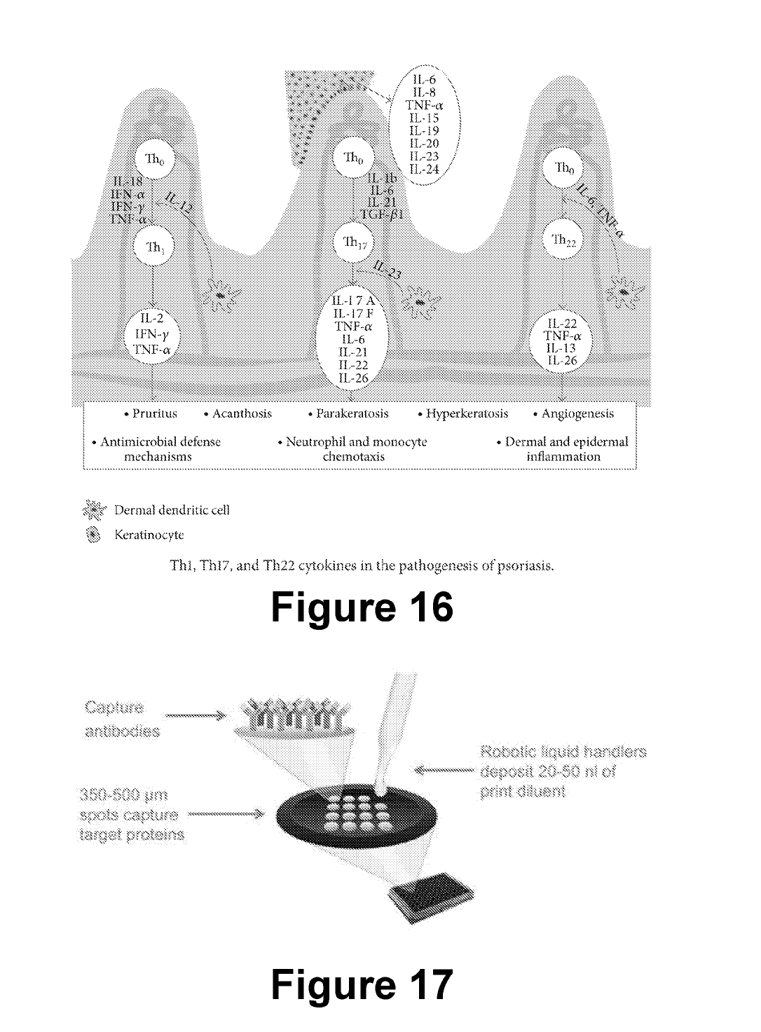

FIG. 16 illustrates Th1, Th17 and Th22 cytokines in the pathogenesis of psoriasis

FIG. 17 illustrates a microarray assay process in accordance with certain embodiments.

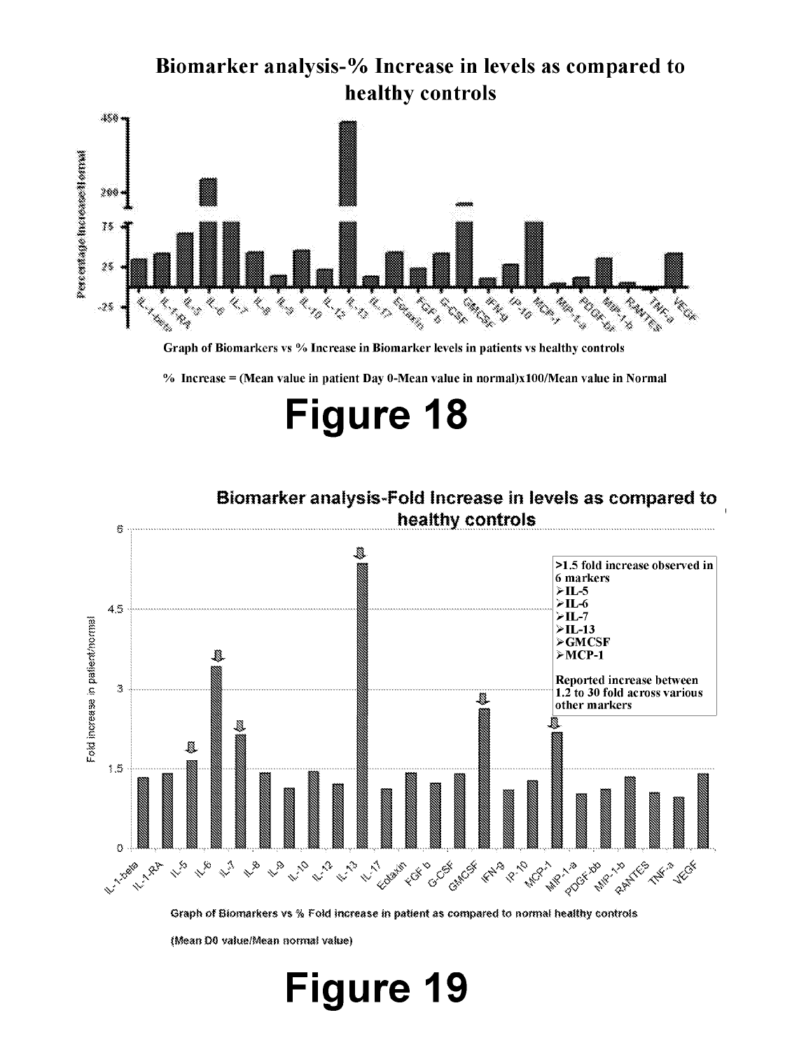

FIG. 18 illustrates % increases in biomarkers levels of psoriasis patients versus healthy controls.

FIG. 19 illustrates % fold increases in biomarker levels of psoriasis patients versus healthy controls.

### Identification of Biomarkers in Serum Samples Collected from Psoriasis Trial on Day 0 and Day 56 by Multiplex Technology

SIRB-001 is a polyherbal mixture of consisting of 3 herbs; Rheum palmatum L. (da huang), Rehmannia glutinosa Libosch (sheng di huang) and Lonicera Japonica (jin yin hua) in ratio of 1:1:3. These individual herbs have been used in Traditional Chinese Medicine for treatment of inflammatory skin disorders.

SIRB-001 has been demonstrated to possess promising anti-psoriatic activity in vitro. Inhibitory effect on keratinocytes proliferation, induction of apoptosis and suppression of inflammatory cytokines has been observed. SIRB-001 has also demonstrated considerable anti-psoriatic potential in TPA and IMQ induced models in vivo.

### Ethnopharmacological Relevance

SIRB-001 is a novel aqueous mixture of 3 Traditional Chinese Medicine (TCM) based herbs; Da Huang (Radix et Rhizoma Rhei), Sheng Di Huang (Radix Rehmanniae) and Jin Yin Hua (Flos Lonicerae) (in the ratio 1:1:3). These constituent herbs are well reported for their multiple biological activities such as anticancer, anti-inflammatory, anti-microbial, immunosuppressive, anti-diabetic and cardiovascular diseases. Psoriasis is a skin disease which is characterized by abnormal, uncontrolled, hyper-proliferation and aberrant differentiation of keratinocytes in the epidermis. This is mediated by a loss of balance between the skin-cells turnover and apoptosis mechanisms resulting in aggravated secretion of pro-inflammatory cytokines. The aim of this study was to investigate the detailed mechanistic action of SIRB-001 in psoriasis by multi-parametric analysis of key markers involved in pathogenesis of this disorder.

The in vitro anti-psoriatic properties of SIRB-001 were assessed by employing HaCaT cells as the test model. Anti-proliferative effect of SIRB-001 (1:500-1:5 v/v) was studied by inhibition of HaCaT cell proliferation using MTT assay. Pro-apoptotic effect of SIRB-001 mediated via early, mid and late markers such as PS externalization (annexin-V staining), mitopotential depolarization (JC-1 staining), caspase-3 activation, cell cycle (SubG0/G1) and DNA fragmentation was examined using flow cytometry and colorimetric methods. Inhibitory effect of SIRB-001 on various pro-inflammatory markers; TNF-α, IFN-γ, TNF-α, IL-6, NO and sPLA₂ was determined against TNF-α stimulated secreted levels by ELISA. VEGF-downregulation in HaCaT cells was studied for its anti-angiogenic potential. Inhibition of IL-17/IL-23 axis, which is crucial in psoriasis, was assessed in immune cells such as murine splenocytes and human monocytic cell line (THP-1). Modulation of key signaling markers such as AKT1 (PKB alpha), FLT3, MAPK1 (ERK2), PRKCA (PKC alpha), MAP2K1 (MEK1), p53 and NFκB was determined. Inhibitory effect of SIRB-001 on Topoisomerase-II activity was determined. In vivo anti-psoriatic effects of SIRB-001 were assessed in TPA induced model in C57BL/6 mice with oral administration and topical application. Resultant inhibitory effects on ear thickness and levels of TNF-α and IL-6 in ear homogenates were determined. Further, SIRB-001 was assessed for anti-psoriatic effects via Inhibitory effect on ear thickness, IL17 and IL-23 secretion in ear homogenates in IMQ induced psoriasis model in BALB/C mice.

Our study demonstrated that SIRB-001 exhibited significant (p<0.01) inhibition of HaCaT cell proliferation after 48 h of treatment (IC₅₀—1:8.8 v/v). This anti-proliferative effect of SIRB-001 correlated well with the noticeable alterations in the cellular morphology and was found to be mediated via significant (p<0.01) activation of all the apoptotic markers tested in HaCaT cells. After 24 h of treatment, SIRB-001 also exhibited significant (p<0.01) anti-inflammatory effect by downregulation of pro-inflammatory markers against TNF-α stimulation and inhibition of VEGF production as compared to basal levels. Alleviation of IL-17/IL-23 in splenocytes and THP-1 cells against Con-A and LPS+IFN-γ stimulated levels respectively was also observed. In addition, SIRB-001 was also able to induce considerable inhibition of kinases and signaling markers, which are intrinsic to the pathology of psoriasis. SIRB-001 demonstrated efficacious anti-inflammatory effects in TPA-induced mice model by reduction of ear thickness and inhibition of TNF-α and IL-6 in ear homogenates against TPA induced levels. Inhibition of ear thickness and IL-23 levels in ear homogenates were also observed in IMQ induced model.

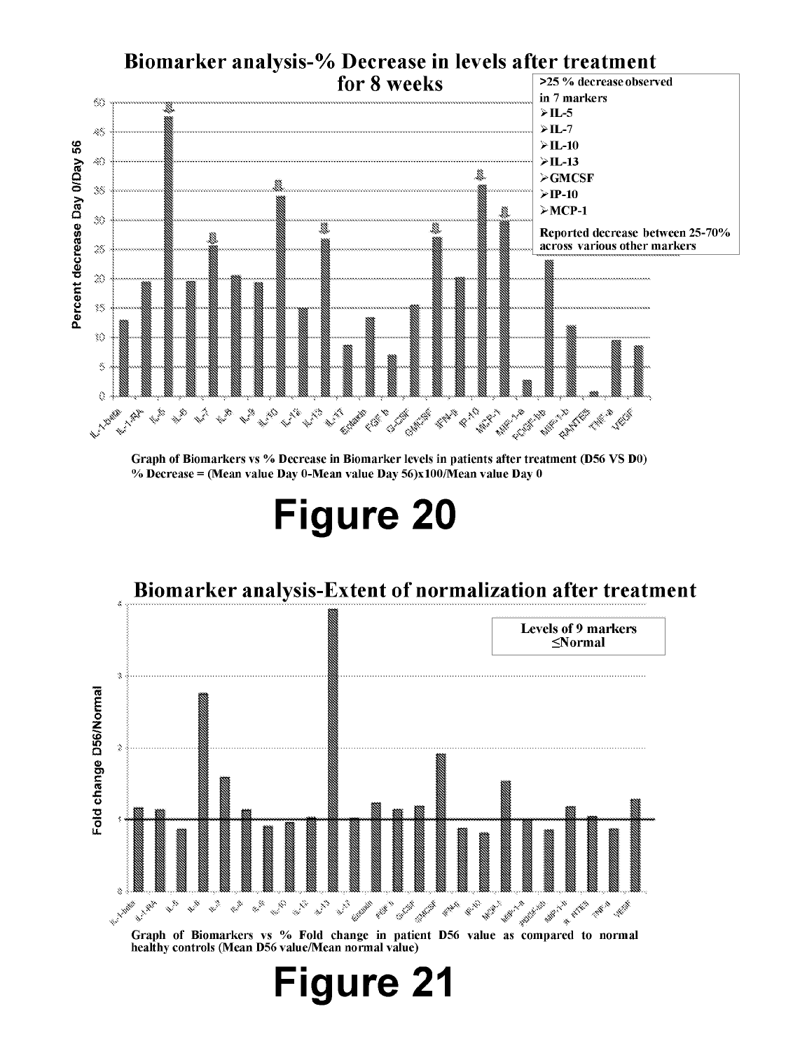

FIG. 20 illustrates % decreases in biomarker levels after eight weeks of treatment. FIG. 21 illustrates the extent of normalization if certain biomarkers after eight weeks of treatment. At least a 20% decrease in levels were observed in 6 markers upon treatment (Comparison between day 0 and day 56) and those markers are the following:

| | |
|---|---|
| 1 | IL-5 |
| 2 | IL-13 |
| 3 | GMCSF |
| 4 | IP-10 |
| 5 | MCP-1 |
| 6 | IL-33 |

Table 5 is an example combination of 6 markers that may be used in a diagnostic or prognostic test kit. Subsets of these 6 markers and other markers may also be used.

The results obtained suggest that SIRB-001exerted anti-psoriatic effects via several mechanisms mediated by anti-proliferative, pro-apoptotic, anti-inflammatory, anti-angiogenic activities and modulation of key signaling markers, which are hallmark features involved in the pathogenesis of psoriasis. SIRB-001 also exhibited efficacious activity models of psoriasis in vivo. Hence, this study highlights the promising potential of SIRB-001 for the management of psoriasis. Based on the above information, SIRB-001 S&L; topical polyherbal hair shampoo & lotion were developed. The clinical safety and efficacy of SIRB-001 S+L, was evaluated in patients with scalp psoriasis in a 8-weeks study.

Strong evidence suggests that immune mechanisms are implicated in the pathogenesis of psoriasis, such as persistent activation of T-lymphocytes and excessive proliferation of keratinocytes. Additionally, cytokines also play a significant role in the pathogenesis of the disease, as they can be found in the affected skin of psoriatic patients. Levels of circulating cytokines and growth factors are also found to be significantly upregulated in serum of psoriasis patients, many of which also correlate with the severity of the disease.

Objective of the present study is to investigate the effect of SIRB-001 S&L, a topical polyherbal hair shampoo & lotion on multiple inflammatory markers in serum samples of psoriatic patients collected at day-0 and day-56, using multiplex technology.

### Conducting Multiplex Analysis in CT1

In the pathogenic processes, biomarkers may serve as surrogate endpoints for diagnosis, prognosis, disease activity, therapeutic response and outcome of the disease.

With the new and now consolidated concepts of “early diagnosis” and “treat to target” a huge effort has been made to identify useful tools to establish early diagnosis, prognosis and therapeutic response.

For this, a great endeavour has been made for the identification of serological, clinical and instrumental parameters, helpful for identify patients at risk of developing persistent and aggressive disease, requiring early and aggressive therapeutic intervention.

There is a real need for developing a platform for identifying biomarkers to aid diagnosis and therapeutic selection, to aid in disease and monitoring and predict side-effect development.

In the era of ever-increasing therapies, a biomarker predicting a favourable response to, and potential side-effects from therapy would eliminate exposure to a drug with potential side-effects and minimal benefit; decrease wastage, and ensure patient satisfaction and increased societal contribution.

### Estimation of Biomarkers by Multiplex

Referring to Table 1, and FIG. 17, thirty five inflammatory markers are associated with psoriasis; eczema; atopic dermatitis or other skin, inflammatory and/or autoimmune ailments, and have been evaluated in patient sera; including multiplex technology being put to use for simultaneous estimation of cytokines. Identification of serological parameters may be helpful to identify patients at risk of developing disease, requiring early and aggressive therapeutic intervention. Working on a non invasive method using patient sera/skin scrapings. Potential for patient stratification in future trials. Potential for developing a prognostic kit/early diagnosis kit.

Biomarker analysis in serum samples of 15 patients and 7 healthy control reveals that levels of 6 biomarkers are upregulated in disease condition.

After 8 weeks of treatment, levels of 8 markers are downregulated from DO value.

Levels of 9 markers seem to be normalized after 8 weeks of treatment (Comparison between D56 value and Normal controls).

Further studies on remaining markers in 15 patients and all markers in 30 patients are in process.

### INCORPORATION BY REFERENCE

What follows is a cite list of references which are, in addition to those references cited above and below herein, and including that which is described as background, the invention summary, brief description of the drawings, the drawings and the abstract, hereby incorporated by reference into the detailed description of the preferred embodiments below, as disclosing alternative embodiments of elements or features of the preferred embodiments not otherwise set forth in detail below. A single one or a combination of two or more of these references may be consulted to obtain a variation of the preferred embodiments described in the detailed description below. Further patent, patent application and non-patent references are cited in the written description and are also incorporated by reference into the preferred embodiments.

A treatment regimen for psoriasis, eczema, inflammation, autoimmune disease, melanoma or other skin ailment, leukemia or other cancer, or other disease including methotrexate, betamethasone or another known treatment described herein, together with administering, before, during and/or after medicinal doses of such known treatment, combinations of the herbs and/or molecules described herein may also be combined with other treatments such as may be understood by those skilled in the art and/or as may be described in literature such as the following which are hereby incorporated by reference, along with the background and brief descriptions of the drawings and priority and related applications, as disclosing alternative embodiments and compounds that may be combined with an herbal and/or molecular combination and a known or discovered treatment or other described treatment in a cocktail or other combinative therapy:

U.S. Pat. Nos. 5,872,103; 6,197,754; 6,740,665; 6,812,255; 7,268,162; 7,358,222; 7,381,535; 7,393,656; 7,563,584; 7,695,926; 7,790,905; 8,541,382; 8,547,695; 8,734,859; and United States published patent applications serial nos. 20030211180; 20050008664;

20050026849; 20050196473; 20060205679; 20070191262; 20080152700; 20080220441; 20090018088; 20090143279; 20090215042; 20090269772; 20100068198; 20100092585; 20100144647; 20100167286; 20120122807; and

PCT published applications no. WO01/66123A2; WO2004/052294A2; WO2006/053049A2; WO2007/130124A1; WO2012/063134A2.

Administration in a treatment regimen of certain combinations with one or two or more of these herbs serve to treat hair and scalp conditions as provided in accordance with embodiments described herein. Specific embodiments include advantageous combinations of Da Huang and Sheng Di Huang, as indicated below and in any one or a combination of U.S. patent application Nos. 61/413,430; 62/325,993; 62/313,709; 62/268,226; 62/259,056; 62/348,762; 15/133,056; 15/131,743; 62/297,796; 62/198,637; 14/754,266; 14/710,865; 14/815,892; 14/287,158; 14/287,153; 13/890,990; 14/981,899; 14/815,705; 13/152,039; PCT/IB11/03078; PCT/IB13/02975; PCT/US15/38341; and US published patent applications nos. 20160051553; 20160136220; 20160136219; 20160136216; 20160136223; 20160136222; 20160136221; 20160113983; 20160143980; 20160113982; 20160136218; 20140205685; and 20140206631; and U.S. Pat. Nos. 9,066,974; 9,095,606; 8,734,859; 8,597,695; and 8,541,382; which are each incorporated by reference, as well as with combinations including Jin Yin Hua with Da Huang and/or Sheng Di Huang. Further embodiments include combinations of Da Huang, Sheng Di Huang and/or Jin Yin Hua with one or more of Mu Dan Pi, Di Gu Pi, Xian He Cao, and/or Chun Gen Pi.

Further embodiments include combinations of beta-sitosterol or saw palmetto, or both, with Da Huang, Sheng Di Huang and/or Jin Yin Hua, and one or more of Mu Dan Pi, Di Gu Pi, Xian He Cao, and/or Chun Gen Pi and/or one or more other herbs or molecules described herein. Further embodiments include herbal combinations of one or more of Sheng Di Huang, Da Huang and Jin Yin Hua with combinations of one or more of emodin, digoxin, beta-sitosterol, saw palmetto, aucubin, rhein, rhapontin, vanillic acid, carvacrol or other herbs or molecules described herein or as understood by those skilled in the art. Other embodiments include combinations including one or more of more of Mu Dan Pi, Di Gu Pi, Xian He Cao, and/or Chun Gen Pi.

Contained within each of the seven herbs are several molecular constituents. Observed reductions of psoriatic inflammation and other studied effects owing to a treatment regimen of periodic shampooing with an herbal formula in accordance with the embodiments can be as a result of various combinations of active molecules contained in Da Huang, Jin Yin Hua and/or Sheng Di Huang, and of combinations of the herbs themselves.

Contained within each of the seven herbs are several molecular constituents. Observed reductions of psoriatic inflammation and other studied effects owing to a treatment regimen of periodic shampooing with an herbal formula in accordance with the embodiments can be as a result of various combinations of active molecules contained in Da Huang, Jin Yin Hua and/or Sheng Di Huang, and of combinations of the herbs themselves.

It is contemplated, as people with ordinary skill in the art would do, that the newly separated compounds may be each individually or in combination used as an ingredient to prepare a pharmaceutical composition for a particular treatment purpose. As it is the status of the art in the pharmaceutical industry, once substantially pure preparations of a compound are obtained, various pharmaceutical compositions or formulations can be prepared from the substantially pure compound using conventional processes or future developed processes in the industry. Specific processes of making pharmaceutical formulations and dosage forms (including, but not limited to, tablet, capsule, injection, syrup) from chemical compounds are not part of the invention and people of ordinary skill in the art of the pharmaceutical industry are capable of applying one or more processes established in the industry to the practice of the present invention. Alternatively, people of ordinary skill in the art may modify the existing conventional processes to better suit the compounds of the present invention. For example, the patent or patent application databases provided at USPTO official website contain rich resources concerning making pharmaceutical formulations and products from effective chemical compounds. Another useful source of information is Handbook of Pharmaceutical Manufacturing Formulations, edited by Sarfaraz K. Niazi and sold by Culinary & Hospitality Industry Publications Services, which is incorporated by reference.

While the invention has been described in terms of several embodiments, those skilled in the art will recognize that the invention is not limited to the embodiments described, but can be practiced with modification and alteration within the spirit and scope of the appended claims. The description is thus to be regarded as illustrative instead of limiting of the invention as set forth in the appended claims including structural and functional equivalents thereof.

1-9. (canceled)

10. A method of treating psoriasis, comprising:
measuring a level of each of IL-13, IL-33 and GMCSF in a first bodily fluid, serum or skin sample extracted from a patient;
formulating a psoriasis diagnosis for the patient based on said measured levels and on one or more expected correlations between said levels and manifestation of psoriasis within said patient; and
administering a medicinal composition to the patient to treat the patient in accordance with said diagnosis.

11. The method of claim 10, further comprising:
measuring a level of each of IL-13, IL-33 and GMCSF in a second bodily fluid, serum or skin sample, extracted from the patient after administering said medicinal composition over a prognostic period; and
indicating to repeat the administering of said medicinal composition to said patient a significant number of further times based on a comparison between said measured levels or combinations of levels within said first and second bodily fluid, serum or skin samples and on an expected correlation between certain differences between said measured levels or combinations of levels in said first and second bodily fluid samples respectively extracted before and after a prognostic period of administration of said medicinal composition.

12. The method of claim 10, wherein the medicinal composition comprises between 20-160 mg/kg of an herbal combination of Da Huang, Sheng Di Huang and Jin Yin Hua.

13-16. (canceled)

17. The method of claim 10, wherein the medicinal composition is formulated as a shampoo, conditioner, cream, lotion, ointment or other topical scalp or hair treatment.

18. The method of claim 10, wherein the medicinal composition is formulated as a cream, lotion, ointment or other topical skin treatment.

19. A medicine for treating psoriasis as in claim 124, said predetermined number of one or more effective doses of said herbal and/or molecular combination are known to decrease by 25% or more over a course of treatment a level of each of: IL-5, IL-13, IL-33, GMCSF, MCP-1, and IP-10.

20-21. (canceled)

22. A medicine for treating psoriasis as in claim 124, wherein said predetermined number of one or more effective doses of said herbal and/or molecular combination are known to decrease by 25% or more over a course of treatment a combination of levels of IL-13, IL-33 and GMCSF.

23. The medicine of claim 22, comprising 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

24. The medicine of claim 22, comprising 1.0 wt. %-15.0 wt. % of an herbal combination of Da Huang, Sheng Di Huang and Jin Yin Hua.

25-27. (canceled)

28. A medical kit, comprising:
a diagnostic kit, including a test kit and an indicator, said test kit for measuring levels of IL-13, IL-33 and GMCSF, in a bodily fluid, serum or skin sample and said indicator for providing a diagnostic result based on said measured levels and on one or more expected correlations between said levels and manifestation of psoriasis; and
a medicinal formulation comprising shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses of an herbal and/or molecular combination known to decrease by 25% or more over a course of treatment levels of each of IL-13, IL-33 and GMCSF.

29. The medical kit of claim 28, further comprising a prognostic kit including a prognostic test kit and a prognostic indicator, said prognostic test kit for measuring levels of each of IL-13, IL-33 and GMCSF, in a second bodily fluid, serum or skin sample, after a prognostic period of treatment, and said prognostic indicator for providing a prognostic result for the patient based on a comparison of said measured levels in the first and second bodily fluid, serum or skin sample.

30. The medical kit of claim 28, wherein said each effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

31. The medical kit of claim 28, wherein said each effective dose comprises between 20-160 mg/kg of an herbal combination of Da Huang, Sheng Di Huang and Jin Yin Hua.

32-53. (canceled)

54. A medicine for treating psoriasis as in claim 124, wherein said predetermined number of one or more effective doses of said herbal and/or molecular combination are known to decrease by 25% or more over a course of treatment a level of each of IL-7, IL-13, IL-33, FGF-β, GMCSF and VEGF.

55-56. (canceled)

57. A medicine for treating psoriasis as in claim 124, wherein predetermined number of one or more effective doses of said herbal and/or molecular combination are known to decrease by 25% or more over a course of treatment a level of each of IL-13 and IL-33.

58-91. (canceled)

92. A medicine for treating psoriasis as in claim 124, wherein said predetermined number of one or more effective doses of said herbal and/or molecular combination are known to decrease by 25% or more over a course of treatment a level of each of IL-33 and GMCSF.

93-123. (canceled)

124. A medicine for treating psoriasis, comprising:
a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses of an herbal and/or molecular combination known to decrease by 25% or more over a course of treatment a level of IL-13, IL-33 or GMCSF, or combinations thereof.

125. The medicine of claim 124, comprising 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

126. The medicine of claim 124, comprising 1.0 wt. %-15.0 wt. % of said herbal combination of Da Huang, Sheng Di Huang and Jin Yin Hua.

127. A medicine for treating psoriasis as in claim 124, wherein said predetermined number of one or more effective doses of said herbal and/or molecular combination is known to decrease by 25% or more over a course of treatment a level of each of IL-13 and GMCSF.

128-140. (canceled)
